(19) United States
(12) Patent Application Publication
(10) Pub. No.: US 2026/0140093 A1
(43) Pub. Date: May 21, 2026

(54) METHODS, COMPOSITIONS AND KITS FOR CONCENTRATING TARGET ANALYTES FROM A BULK FLUID SAMPLE

(71) Applicant: PHASE SCIENTIFIC INTERNATIONAL, LTD., Hong Kong (CN)

(72) Inventors: Vasu SAINI, Hong Kong (CN); Cheuk Yiu Tenny CHUNG, Hong Kong (CN); Daniel William BRADBURY, Hong Kong (CN); Harsha Madan KITTUR, Singapore (SG); Masae Kobayashi WEN, Buena Park, CA (US); Wai Sum TSANG, Hong Kong (CN); Yin To CHIU, Hong Kong (CN); Garrett Lee MOSLEY, Hong Kong (CN)

(21) Appl. No.: 19/123,391

(22) PCT Filed: Sep. 8, 2023
(86) PCT No.: PCT/CN2023/117657

§ 371 Date: Apr. 22, 2025

Related U.S. Application Data
(60) Provisional application No. 63/381,932, filed on Nov. 2, 2022.

(51) Int. Cl. G01N 30/06; G01N 1/40; G01N 30/34; G01N 33/50

(52) U.S. Cl. CPC G01N 30/06; G01N 1/40; G01N 30/34; G01N 33/50; G01N 2800/34; G01N 2800/50; G01N 2800/7028

(57) ABSTRACT

The present disclosure relates to methods, compositions, and kits for concentrating and purifying one or more target analyte (s) from a bulk fluid sample. In some embodiments, the methods involve at least two first aqueous two-phase system (ATPS) compositions. Some embodiments provide a kit comprising at least two ATPS compositions, and a binding buffer. Other embodiments provide methods of treating bladder cancer in a patient in need thereof.

### CROSS-REFERENCE TO RELATED APPLICATION

This application claims priority to, and the benefits of, U.S. Provisional Application having Ser. No. 63/381,932 filed on Nov. 2, 2022. The entire contents of the foregoing application are hereby incorporated by reference in its entirety for all purposes.

### FIELD OF INVENTION

This application relates to methods, compositions and kits for improving the concentration and purification of target analytes using an aqueous two-phase system (ATPS), and in particular, methods, compositions and kits for improving the concentration and purification of target analytes from a bulk fluid sample.

### BACKGROUND OF INVENTION

It is a challenging task to efficiently concentrate and isolate target analytes from a large amount of biological sample for downstream applications, such as for diagnostic tests. Accordingly, there is a need for improved methods that are simpler, less expensive, and can quickly handle large amounts of samples to provide high quality target analytes.

### SUMMARY OF INVENTION

Disclosed herein are novel methods, compositions and kits that are useful for isolation, concentration and/or purification of target analytes, such as nucleic acids, employing aqueous two-phase systems (ATPS) and a purification system, such as an extraction column, without the need for complex equipment.

In some embodiments, provided is a method for concentrating and purifying one or more target analyte (s) from a bulk fluid sample, including the steps of: (a) dividing the bulk fluid sample containing the target analyte (s) into at least two aliquots of a sample solution; (b) preparing at least two first aqueous two-phase system (ATPS) compositions, wherein each first ATPS composition includes a polymer, a salt, a surfactant, or any combination thereof dissolved in an aqueous solution to form a first phase solution and a second phase solution; (c) adding each aliquot of said sample solution prepared from the bulk fluid sample containing the target analyte (s) to each first ATPS composition, such that the target analyte (s) partition to each first phase solution; (d) further processing each first phase solution to form a final phase solution; (e) mixing the final phase solution with at least one purifying composition to form a mixed solution; (f) contacting the mixed solution with a purification system configured to selectively isolate the target analyte (s); and (g) collecting the target analyte (s) from the purification system, resulting in a final solution containing the concentrated and purified target analyte (s).

In some embodiments, provided is a method for concentrating and purifying one or more target analytes from a bulk fluid sample, including the steps of: (a) dividing the bulk fluid sample containing the target analyte (s) into at least two aliquots of a sample solution; (b) preparing at least two aliquots of a first aqueous two-phase system (ATPS) composition, wherein the first ATPS composition includes polymers, salts, surfactants, or combinations thereof dissolved in an aqueous solution to form a first phase solution and a second phase solution; (c) adding each aliquot of said sample solution containing the target analyte (s) to each aliquot of the first ATPS composition, such that the target analyte (s) partition to the first phase solution; (d) collecting the first phase solutions of the at least two aliquots of the first ATPS composition, and mixing the first phase solutions with a second ATPS composition, wherein the second ATPS composition includes polymers, salts, surfactants, or combinations thereof dissolved in an aqueous solution to form a third phase solution and a fourth phase solution, such that the target analyte (s) partition to and concentrate in the third phase solution; (e) collecting the third phase solution and mixing the third phase solution with a binding buffer to form a mixed solution, wherein the binding buffer includes at least one chaotropic agent; (f) loading the mixed solution onto an extraction column configured to selectively extract and purify the target analyte (s); (g) eluting and collecting the target analyte (s) from the extraction column.

Another embodiment provides an ATPS composition comprising one or more polymers, salts, surfactants, or combinations thereof, as described herein.

In some embodiments, provided is an ATPS composition selected from the group consisting of A1, A2, A3, A4, AA1, AA2, AA3, and AA4.

In some embodiments, provided is a kit including a first ATPS composition selected from the group consisting of A1, A2, A3, and A4; a second ATPS composition selected from the group consisting of AA1, AA2, AA3, and AA4; and a binding buffer selected from the group consisting of B1, B2, and B3.

In some embodiments, provided is a method for concentrating and purifying one or more target analytes from a bulk fluid sample, comprising the steps of:
(a) preparing a first aqueous two-phase system (ATPS) composition, wherein the first ATPS composition comprises polymers, salts, surfactants, or combinations thereof dissolved in an aqueous solution to form a first phase solution and a second phase solution;
(b) adding a sample solution prepared from the bulk fluid sample containing the target analyte (s) to the first ATPS composition, such that the target analyte (s) partition to the first phase solution;
(c) collecting the first phase solution and mixing the first phase solution with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution;
(d) collecting the third phase solution and mixing the third phase solution with a binding buffer to form a mixed solution, wherein the binding buffer comprises at least one chaotropic agent;
(e) loading the mixed solution onto an extraction column configured to selectively extract and purify the target analyte (s);
(f) eluting and collecting the target analyte (s) from the extraction column, resulting in a final solution containing the concentrated and purified target analyte (s).

In some embodiments, provided is a method for concentrating and purifying one or more target analytes from a bulk fluid sample, comprising the steps of:
(a) dividing the bulk fluid sample containing the target analyte (s) into at least two aliquots of a sample solution;
(b) preparing at least two aliquots of a first aqueous two-phase system (ATPS) composition, wherein the first ATPS composition comprises polymers, salts, surfactants, or combinations thereof dissolved in an aqueous solution to form a first phase solution and a second phase solution;
(c) adding each aliquot of said sample solution containing the target analyte (s) to each aliquot of the first ATPS composition, such that the target analyte (s) partition to the first phase solution;
(d) collecting the first phase solutions of the at least two aliquots of the first ATPS composition, and mixing the first phase solutions with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution;
(e) collecting the third phase solution and mixing the third phase solution with a binding buffer to form a mixed solution, wherein the binding buffer comprises at least one chaotropic agent;
(f) loading the mixed solution onto an extraction column configured to selectively extract and purify the target analyte (s);
(g) eluting and collecting the target analyte (s) from the extraction column, resulting in a final solution containing the concentrated and purified target analyte (s).

In some embodiments, a respective kit can be advantageously used in conjunction with and for performing the methods according to the various aspects of the invention. In some embodiments, the kit may include the components described in the various embodiments, but may additionally include syringe or pipette accessible containers for storage, packing, and/or reactions and optionally equipment for manipulating the aqueous solutions. Such containers and equipment may include columns, test tubes, capillary tubes, plastic test tubes, falcon tubes, culture tubes, well plates, pipettes, cuvettes or the like.

Other example embodiments are discussed herein.

### Advantages

There are many advantages to the various embodiments of the present disclosure.

In some embodiments, the methods, compositions and kits of the present disclosure surprisingly and effectively concentrate and isolate target analytes from large amounts of liquid biological samples. The methods, compositions and kits are particularly effective at isolating target analytes that exist at very small concentrations in the biological sample, such as cell-free DNA (cfDNA). The methods, compositions and kits allow for more accurate detection and identification of target analytes from a large volume of liquid, which is particularly useful for diagnostic purposes. These methods, compositions and kits are particularly useful for use in biological samples that typically come in large volumes, such as urine, saliva, blood, and others.

In some embodiments, the methods, compositions and kits of the present disclosure provide simple, less expensive, and efficient means to purify target analytes from different clinical/biological samples of different volumes, especially in processing large volume or bulk fluid samples to provide high quality target analytes. The methods. compositions and kits disclosed herein involve Aqueous Two-Phase Systems (ATPS) in the upstream process, providing great flexibility for use with a variety of downstream processes.

In some embodiments, it is desirable to split large volume or bulk fluid samples into smaller aliquots for a multitude of reasons. For example, multiple smaller aliquots can be processed in parallel (also referred to as “parallel ATPS” in some embodiments) in order to save time, to minimize the use of reagents, to enable less extreme volume ratio in the ATPS, and to accommodate the sample size limit of the available instruments.

In some embodiments, it is surprisingly found that although the disclosed methods include additional steps (such as dividing the bulk fluid sample into at least two aliquots, performing phase separation using multiple ATPS in parallel, etc.) which are potential sources for loss of target analyte (e.g. due to imperfect target partitioning in ATPS), the recovery efficiency of target analytes using the disclosed methods shows no significant difference when compared to the recovery efficiency using a method with a single ATPS to process the bulk fluid sample.

As such, the methods and kits of the present disclosure can be adapted by any persons skilled in the art in different laboratory and equipment settings to achieve comparable DNA recovery from large volume or bulk fluid samples while minimizing errors and sample loss typically associated with sample handling and processing.

In some embodiments, a large clinical/biological sample volume results in a large target-rich phase in the first ATPS. In some embodiments, a second ATPS is used to concentrate the large target-rich phase of the first ATPS into a more concentrated, smaller volume of a target-rich phase in the second ATPS for more user-friendly downstream processing.

In some embodiments, having a second ATPS following the first ATPS, DNA can be further concentrated for detection.

These and other features and characteristics, as well as the methods of operation and functions of the related components, will become more apparent upon consideration of the following detailed description and the appended claims with reference to the accompanying figures, all of which form a part of this specification, wherein like reference numerals designate corresponding parts in the various figures. It is to be expressly understood, however, that the figures are for the purpose of illustration and description only and are not intended as a definition of the limits of the claims.

### BRIEF DESCRIPTION OF FIGURES

FIG. 1A is a graph showing the average CT values of 145 bp DNA recovered from urine using spin column with and without prior ATPS steps according to an example embodiment.

FIG. 1B is a graph showing the average CT values of 2000 bp DNA recovered from urine using spin column with and without prior ATPS steps according to an example embodiment.

FIG. 1C is a graph showing the average CT values of 145 bp DNA recovered from urine using magnetic beads with and without prior ATPS steps according to an example embodiment.

FIG. 1D is a graph showing the average CT values of 2000 bp DNA recovered from urine using magnetic beads with and without prior ATPS steps according to an example embodiment.

FIG. 2A is a graph showing the average CT values of 145 bp DNA recovered from urine under the different extraction conditions according to Table 3.

FIG. 2B is a graph showing the average CT values of 145 bp DNA recovered from urine under different extraction conditions according to Table 6.

FIG. 3A is a graph showing the average CT values of 145 bp DNA recovered from urine samples from three individual donors using 1st ATPS with varying top:bottom phase volume ratio, according to Table 9.

FIG. 3B is a graph showing the average CT values of 145 bp DNA recovered from 0.25×PBS as sample matrix using 1st ATPS with varying top:bottom phase volume ratio, according to Table 9.

FIG. 3C is a graph showing the average CT values of 145 bp DNA recovered from urine samples from three individual donors using 2nd ATPS with varying top:bottom phase volume ratio, according to Table 10.

FIG. 3D is a graph showing the average CT values of 145 bp DNA recovered from 0.25×PBS as sample matrix using 2nd ATPS with varying top:bottom phase volume ratio, according to Table 10.

FIG. 4A is a graph showing the recovery of 145 bp DNA spike-in (copies/uL) using the conditions A-F according to Table 15.

FIG. 4B is a graph showing the average concentration of recovered DNA (copies/uL) using the kits and conditions according to Table 16.

### DETAILED DESCRIPTION

Unless indicated otherwise, the terms used herein, including technical and scientific terms, have the same meaning as usually understood by those skilled in the art to which the present invention pertains and detailed descriptions of well-known functions and constitutions that may obscure the gist of the present invention are omitted.

As used herein and in the claims, “comprising” and “including” mean including the following elements but not excluding others.

As used herein and in the claims, the terms “comprising” (or any related form such as “comprise” and “comprises”), “including” (or any related forms such as “include” or “includes”), “containing” (or any related forms such as “contain” or “contains”), or “having” (or any related forms such as “have” or “has”) means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” “containing,” or “having” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “asolution comprising A, B, and C” would be “asolution consisting of A, B, and C” and “asolution consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

“Essentially consisting of” limits the scope of a claim to the specified materials, components, or steps (“essential elements”) that do not materially affect the essential characteristic (s) of the claimed invention. In some embodiments, the essential characteristics are the basic and novel characteristic (s) of the claimed invention.

As used herein, the singular forms “a”, “an” and “the” are intended to include the plural forms as well, unless the context clearly indicates otherwise. Where a range is referred in the specification, the range is understood to include at least each discrete point within the range. For example, 1-7 in some embodiments means 1, 2, 3, 4, 5, 6, and 7. Unless otherwise indicated, a range is meant to include all values that fall within the range, including whole numbers, fractions, portions, and the like. For example, a range of 1-7 when described in a claim refers to a scope that includes values and sub-ranges such as 1, 1.5, 2-3, 6, and 7, by way of example.

As used herein, the term “about” is understood as within a range of normal tolerance in the art and not more than ±10% of a stated value. By way of example only, about 50 means from 45 to 55 including all values in between. As used herein, the phrase “about” a specific value also includes the specific value, for example, about 50 includes 50.

“Aqueous,” as used herein, refers to the characteristic properties of a solvent/solute system wherein the solvating substance has a predominantly hydrophilic character. Examples of aqueous solvent/solute systems include those where water, or compositions containing water, are the predominant solvent. The polymer and/or surfactant components whose use is described in the embodiments are “aqueous” in the sense that they form aqueous phases when combined with a solvent such as water. Further, as understood by the skilled person, in the present context the term liquid “mixture” refers merely to a combination of the herein-defined components.

As used herein, an aqueous two-phase system (ATPS) means a liquid-liquid separation system that can accomplish isolation or concentration of an analyte by partitioning, where two phases, sections, areas, components, or the like, interact differently with at least one analyte to which they are exposed and optionally dissolved. An ATPS is formed when two immiscible phase forming components, such as a salt and polymer, or two incompatible polymers (e.g., PEG and dextran) with certain concentrations are mixed in an aqueous solution. ATPS methods are relatively inexpensive and scalable because they employ two-phase partitioning to separate analytes (e.g., nucleic acids) from contaminants.

The term ‘isolated’ as used herein refers to an analyte being removed from its original environment and thus is altered from its original environment. For example, an isolated nucleic acid generally is provided with fewer non-nucleic acid components (e.g., protein, lipid) than the amount of components present in a source sample. A composition comprising an isolated analyte, (e.g., sample nucleic acid) can be substantially isolated (e.g., about 90%, 91%, 92%, 93%, 94%, 95%, 96%, 97%, 98%, 99% or greater than 99% free of non-analyte components (such as non-nucleic acid components)).

As used herein, “concentrated” means that the mass ratio of analyte in question to the solution in which the analyte is suspended is higher than the mass ratio of said analyte in its pre-concentration solution. It can, for example, be slightly higher, or more preferably at least twice, ten times or one hundred times as high.

As used herein, the term “downstream purification system” refers to a device, a method or a process to purify and selectively isolate the target analyte by chemical or physical means. In some embodiments, a downstream purification system comprises a solid phase medium, wherein the solid phase medium is a solid phase extraction column. In some embodiments, the solid phase extraction column is a spin column. In some embodiments, the solid phase medium is a plurality of beads, silica resins, silica membrane, silica gel, alumina gel, size exclusion resins, or ion-exchange resins. In some embodiments, a downstream purification system is a method or process which includes a step of precipitating the target analyte from a purifying composition. In some embodiments, the purifying composition comprises alcohol.

As used herein, the terms “flow-through”, “flow through” and “supernatant” all refer to the liquid or solution that passes through or separates from the solid phase medium, which can be removed or isolated from the solid phase medium. In some embodiments, supernatant refers to the flow-through that passes through a column.

As used herein, the terms “perturbing” or “perturbation” refers to the process of introducing physical force and disturbance into a provided system. In some embodiments, perturbing a solid phase extraction complex introduces centrifugation force, magnetic force, or combination thereof, which causes separation of target analyte (s) from or into the solid phase medium or supernatant. In some embodiments, examples of perturbing or perturbation are, but not limited to, centrifuging, vacuuming, magnetizing, vortexing, spinning, swirling, rotating, shaking, stirring, rocking, and combinations thereof. In some embodiments, centrifuging or vortexing is achieved by using a centrifuging machine or a vortex. In some embodiments, vacuuming means contacting the solid phase extraction complex to a vacuum manifold to result in a flow-through or supernatant. In some embodiments, perturbation such as magnetizing, spinning, swirling, rotating, shaking, stirring, and rocking is achieved manually or by an appropriate instrument. In some embodiments, centrifuging and magnetizing are performed simultaneously.

As used herein, “cell-free DNA” (cfDNA) is DNA that is present outside a cell, e.g., DNA present in the sample (e.g. blood, plasma, serum, or urine) obtained from a subject.

As used herein, the term “polymer” refers to any polymer including at least one substituted or non-substituted monomer. Examples of polymer includes, but are not limited to, homopolymer, copolymer, terpolymer, random copolymer, and block copolymer. Block copolymers include, but are not limited to, block, graft, dendrimer, and star polymers. As used herein, copolymer refers to a polymer derived from two monomeric species; similarly, a terpolymer refers to a polymer derived from three monomeric species. The polymer also includes various morphologies, including, but not limited to, linear polymer, branched polymer, random polymer, crosslinked polymer, and dendrimer systems. In some embodiments, a polymer also includes its chemically modified equivalent, such as hydrophobically-modified, or silicone-modified. As an example, polyacrylamide polymer refers to any polymer including at least one substituted or non-substituted acrylamide unit, e.g., a homopolymer, copolymer, terpolymer, random copolymer, block copolymer or terpolymer of polyacrylamide; polyacrylamide can be a linear polymer, branched polymer, random polymer, crosslinked polymer, or a dendrimer of polyacrylamide; polyacrylamide can be hydrophobically-modified polyacrylamide, or silicone-modified polyacrylamide.

In some embodiments, examples of polymer include, but are not limited to, polyethers, polyimines, polyalkylene glycols, vinyl polymers, alkoxylated surfactants, polysaccharides, polyether-modified silicones, polyacrylamides, polyacrylic acids and copolymers thereof. In some embodiments, the polymer is hydrophobically-modified, or silicone-modified.

Examples of polyalkylene glycols (also referred as ‘PAG’ or ‘poly(oxyalkylene)’ or ‘poly(alkylene oxide)’) include, but are not limited to, hydrophobically modified polyalkylene glycols, poly (oxyalkylene) polymer, poly (oxyalkylene) copolymer, hydrophobically modified poly (oxyalkylene) copolymers, dipropylene glycol, tripropylene glycol, polyethylene glycol (also referred as ‘PEG’), polypropylene glycol (also referred as ‘PPG’). In some embodiments, examples of copolymers of PAGs include, but are not limited to, poly (ethylene glycol-propylene glycol) (also referred as ‘PEG-PPG’ or ‘UCON’), and poly (ethylene glycol-ran-propylene glycol) (also referred as ‘PEG-ran-PPG’). In some embodiments, PEG-PPG comprises random copolymers, block copolymers, or combination thereof. In some embodiments, PEG-PPG comprise both random copolymers and block copolymers. In some embodiments, PEG-PPG is PEG-ran-PPG.

As used herein, “vinyl polymer” refers to a group of polymers derived from substituted vinyl (H₂C═CHR) monomers. Examples of vinyl polymer include, but are not limited to, polyvinyl pyrrolidone, polyvinyl alcohol, polyvinyl caprolactam, and polyvinyl methylether.

Examples of polysaccharides include, but are not limited to, dextran, carboxymethyl dextran, dextran sulfate, hydroxypropyl dextran, starch, carboxymethyl cellulose, hydroxypropyl cellulose, methyl cellulose, ethylhydroxyethylcellulose, and maltodextrin. In some embodiments, polysaccharides are alkoxylated starches, alkoxylated cellulose, or alkyl hydroxyalkyl cellulose.

Examples of polyacrylamides include, but are not limited to, poly N-isopropylacrylamide.

Examples of polyimines include, but are not limited to, polyethyleneimine.

Examples of alkoxylated surfactants include, but are not limited to, carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, sodium N-lauroyl sarcosinate (NLS), ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, and polyoxyethylene fatty acid amides.

In some embodiments, the polymer has an average molecular weight of about 200-1,000 Da, 200-35,000 Da, 300-35,000 Da, 400-2,000 Da, or 400-35,000 Da. Examples thereof include, but are not limited to, polyalkylene glycols (PAGs) with average molecular weight of about 400 Da, 500 Da, 600 Da, 700 Da, 800 Da, 900 Da, 1,000 Da, 2,000 Da, 3,000 Da, 4,000 Da, 5,000 Da, 6,000 Da, 7,000 Da, 8,000 Da, 9,000 Da, 10,000 Da, 15,000 Da, 20,000 Da, 25,000 Da, 30000 Da, and 35000 Da. In some embodiments, the PAG has an average molecular weight at a range of between any of the two molecular weights listed above.

Examples of PAG include, but are not limited to PEG 200, PEG 300, PEG 400, PEG 500, PEG 600, PEG 700, PEG 800, PEG 900, PEG 1000, PEG 2000, PEG 3000, PEG 4000, PEG 5000, PEG 6000, PEG 7000, PEG 8000, PEG 9000, PEG 10000, PEG 15000, PEG 20000, PEG 25000, PEG 30000, PEG 35000, PPG 425, PPG 725, PPG 900, PPG 1000, and PPG 2000. In some embodiments, the PEG has an average molecular weight at a range of between any of the two PEG molecular weights listed above. In some embodiments, the PPG has an average molecular weight at a range of between any of the two PPG molecular weights listed above.

In some embodiments, the polymer comprises ethylene oxide (EO) and propylene oxide (PO) units, and has an ethylene oxide:propylene oxide (EO:PO) ratio of 90:10 to 10:90. In some embodiments, the polymer has an EO:PO ratio of 10:90, 15:85, 20:80, 25:75, 30:70, 35:65, 40:60, 45:55, 50:50, 55:45, 60:40, 65:35, 70:30, 75:25, 80:20, 85:15, or 90:10. In some embodiments, the polymer has an EO:PO ratio at a range between any of the two ratios listed above.

In some embodiments, the polymer is a PAG having an average molecular weight of about 980-12,000 Da and an EO:PO ratio of 50:50 to 75:25. Examples thereof include, but are not limited to, PEG-PPGs with average molecular weight of about 980 Da, 1,230 Da, 1,590 Da, 2,470 Da, 2,660 Da, 3,380 Da, 3,930 Da, 6,950 Da, and 12,000 Da. In some embodiments, the PEG-PPGs has an average molecular weight at a range of between any of the two PEG-PPGs molecular weights listed above. In some embodiments, PEG-PPG comprises an EO:PO ratio of 50:50, or 75:25. In some embodiments, the polymer is PEG-ran-PPG with an average molecular weight of about 2,500 or 12,000 Da and having an EO:PO ratio of about 75:25.

In some embodiments, the polymer is a vinyl polymer having an average molecular weight of about 2,500-2,500,000 Da. Examples thereof include, but are not limited to polyvinyl pyrrolidone with an average molecular weight of about 2,500 Da, 10,000 Da, 40,000 Da, 100,000 Da, and 2,500,000 Da. In some embodiments, the vinyl polymer has an average molecular weight at a range of between any of the two molecular weights listed above.

In some embodiments, the polymer is a polysaccharide and has an average molecular weight from about 6,000-5,000,000 Da. Examples thereof include, but are not limited to dextrans with average molecular weight of about 6,000 Da, 12,000 Da, 25,000 Da, 60,000 Da, 70,000 Da, 80,000 Da, 150,000 Da, 270,000 Da, 410,000 Da, 450,000 Da, 550,000 Da, 650,000 Da, 670,000 Da, 1,500,000 Da, 2,000,000 Da, 2, 800,000 Da, 4,000,000 Da and 5,000,000 Da. In some embodiments, the dextran has an average molecular weight at a range of between any of the two molecular weights listed above.

In some embodiments, the polymer is a polyether and has an average molecular weight of about 200-35,000 Da. Examples thereof include, but are not limited to silicon modified polyether (or ‘polyether-modified silicones’) with average molecular weight of about 200 Da-35,000 Da.

In some embodiments, the polymer is a polyacrylamide and has an average molecular weight of 1,000-5,000,000 Da. Examples thereof include, but are not limited to polyacrylamide or poly (N-isopropylacrylamide) with average molecular weight of 1,000 Da, 2,000 Da, 5,000 Da, 10,000 Da, 40,000 Da, 85,000 Da, 5,000,000 Da. In some embodiments, the polyolefin has an average molecular weight at a range of between any of the two molecular weights listed above.

In some embodiments, the polymer is a polyacrylic acid and has an average molecular weight of about 1,250-4,000,000 Da. Examples thereof include, but are not limited to, polyacrylic acids with average molecular weight of 1,200 Da, 2, 100 Da, 5, 100 Da, 8,000 Da, 8, 600 Da, 8, 700 Da, 16,000 Da, and 83,000 Da. In some embodiments, the polyolefin has an average molecular weight at a range of between any of the two molecular weights listed above.

As used herein, the term “salt” refers to a substance having at least one cation and at least one anion. Examples of salts include, but are not limited to, salts wherein the cation is sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium or tetrabutyl ammonium, and/or wherein the anion is phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, or tris. In some embodiments, the salts are kosmotropic salts, chaotropic salts, or inorganic salts.

As used herein, examples of “surfactant” include, but are not limited to, anionic surfactant, nonionic surfactant, cationic surfactant, zwitterionic surfactant or amphoteric surfactant.

Examples of anionic surfactants include, but are not limited to, carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, and sodium N-lauroyl sarcosinate (NLS).

Examples of nonionic surfactants include, but are not limited to, ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, and polyoxyethylene fatty acid amides.

Examples of cationic surfactants include, but are not limited to, quaternary ammonium salts, amines with amide linkages, polyoxyethylene alkyl amines, polyoxyethylene alicyclic amines, n, n, n′, n′tetrakis substituted ethylenediamines, and 2-alkyl 1-hydroxethyl 2-imidazolines,

Examples of amphoteric surfactants include, but are not limited to, n-coco 3-aminopropionic acid or sodium salt thereof, n-tallow 3-iminodipropionate or disodium salt thereof, n-carboxymethyl n dimethyl n-9 octadecenyl ammonium hydroxide, n-cocoamidethyl n hydroxyethylglycineor sodium salt thereof, and sodium N-lauroyl sarcosinate (NLS).

In some embodiments, the surfactant comprises a polymer such as PAG. In some embodiments, the surfactant has a structure of EOₓ-PO_{y}-EOₓ, wherein EO refers to an ethylene oxide unit and PO refers to a propylene oxide unit, and x and y are the respective number of monomers. In some embodiments, x=2-136. In some embodiments, y=16-62. In some embodiments, examples of surfactants include, but are not limited to, (C₂H₄O)ₙCl₄H₂₂O wherein n=4-10 (such as Triton X-100, Triton X-114, Triton X-45, Tween 20, Igepal CA630), Brij 58, Brij O10, Brij L23, EOₓ-PO_{y}-EOₓ wherein x=2-136 and y=16-62 (such as Pluronic L-61, Pluronic F-127), UCON, sodium dodecyl sulfate, sodium cholate, sodium deoxycholate, N-lauroyl sarcosine sodium salt (NLS), hexadecyltrimethlammonium bromide, or span 80.

In some embodiments, the target analyte is a nucleic acid, a protein, an antigen, a biomolecule, a sugar moiety, a lipid, a sterol, exosomes, or any combination thereof. In some embodiments, examples of target analyte include, but are not limited to, genomic DNA (gDNA), cDNA, plasmid DNA, mitochondrial DNA, cell-free DNA (cfDNA), circulating tumor DNA (ctDNA), circulating fetal DNA, cell-free microbial DNA, micro RNA (miRNA), messenger RNA (mRNA), transfer RNA (tRNA), ribosomal RNA (rRNA), circular RNA, long non-coding RNA (lncRNA) or combinations thereof.

As used herein, “biological sample” refers to any tangible material obtained directly or indirectly from an organism, such as a virus, bacterium, plant, animal, or human Examples of biological samples include but are not limited to nucleic acids, proteins, cells, cellular organelles, tissue extracts, tissues, organs, biofluids such as blood, plasma, urine, saliva, stool, cerebrospinal fluid (CSF), lymph, serum, sputum, peritoneal fluid, sweat, tears, nasal swab, vaginal swab, endocervical swab, semen, breast milk, and other bodily fluids.

As used herein, “clinical sample” refers to any sample obtained directly or indirectly from a subject (e.g., a human). In some embodiments the subject is a human patient. Examples of clinical samples include but are not limited to blood, plasma, urine, saliva, stool, cerebrospinal fluid (CSF), lymph, serum, sputum, peritoneal fluid, sweat, tears, nasal swab, vaginal swab, endocervical swab, semen, breast milk, and other bodily fluids.

The term “large volume”, “large amount”, “high volume”, or “bulk fluid”, “bulk fluid sample” when referring to liquid samples in the present disclosure means a biological sample that has a volume of at least 1 mL, 2 mL, 3 mL, 4 mL, 5 mL, 6 mL, 7 mL, 8 mL, 9 mL, 10 mL, 20 mL, 30 mL, 40 mL, 50 mL, 60 mL, 70 mL, 80 mL, 90 mL, 100 mL, 200 mL, 300 mL, 400 mL, 500 mL. In some embodiments, the sample has a volume of 1-5 mL, 1-10 mL, 15-20 mL, 10-20 mL, 20-30 mL, or 30-40 mL. In some embodiments, the sample has a volume of at least 40 mL. In some embodiments, the sample has a volume range of 10 mL-40 mL, 10 mL-50 mL, 10 mL-100 mL; 40 mL-50 mL, 40 mL-60 mL, 40 mL-100 mL, 40 mL-160 mL, 40 mL-200 mL, 50 mL-100 mL, 50 mL-200 mL, or 50 mL-300 mL. In some embodiments, the sample has a volume of at least 10 mL, 20 mL, 30 mL, 40 mL, 50 mL, 60 mL, 70 mL, 80 mL, 90 mL, or 100 mL; and at most 100 mL, 200 mL, 300 mL, 400 mL, or 500 mL.

As used herein, the term “Ct”, “CT”, “Ct value” or “CT value” refers to a cycle threshold value and signifies the cycle of a PCR amplification assay in which signal from a reporter that is indicative of amplicon generation (e.g., fluorescence) first becomes detectable above a background level. In some embodiments, the CT value is an indirect indicator of the amount of target nucleic acid detected from a particular sample. In general, a lower CT value indicates a higher amount of the target nucleic acid in the sample, and a higher CT value indicates a lower amount of the target nucleic acid in the sample.

As used herein, the term “chaotropic agent” refers to a substance that disrupts the hydrogen bonding network between water molecules in solution. In some embodiments, the chaotropic agent is a thiocyanate, isothiocyanate, perchlorate, acetate, trichloroacetate, trifluoroacetate, chloride, or iodide. Examples of chaotropic agents include, but are not limited to, guanidinium hydrochloride (GHCl), guanidinium thiocyanate, guanidinium isothiocyanate (GITC)), sodium thiocyanate, sodium iodide, sodium perchlorate, sodium trichloroacetate, sodium trifluroacetate, lithium perchlorate, lithium acetate, magnesium chloride, phenol, 2-propanol, thiourea, urea and the like.

### EMBODIMENTS OF THE PRESENT INVENTION

### Embodiment 1

One aspect provides a method for concentrating and purifying one or more target analytes from a bulk fluid sample, comprising the steps of:
(a) preparing a first aqueous two-phase system (ATPS) composition, wherein the first ATPS composition comprises polymers, salts, surfactants, or combinations thereof dissolved in an aqueous solution to form a first phase solution and a second phase solution;
(b) adding a sample solution prepared from the bulk fluid sample containing the target analyte (s) to the first ATPS composition, such that the target analyte (s) partition to the first phase solution;
(c) collecting the first phase solution and mixing the first phase solution with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution;
(d) collecting the third phase solution and mixing the third phase solution with a binding buffer to form a mixed solution, wherein the binding buffer comprises at least one chaotropic agent;
(e) loading the mixed solution onto an extraction column configured to selectively extract and purify the target analyte (s);
(f) eluting and collecting the target analyte (s) from the extraction column, resulting in a final solution containing the concentrated and purified target analyte (s).

In some embodiments, the sample solution is prepared by dividing the bulk fluid sample containing the target analyte (s) into at least two aliquots of the sample solution, and the first ATPS composition is divided into at least two aliquots, wherein step (b) further includes the following steps:
(i) adding each aliquot of said sample solution prepared from the bulk fluid sample containing the target analyte (s) to each aliquot of the first ATPS composition, such that the target analyte (s) partition to the first phase solution;
(ii) collecting and combining the first phase solutions of the at least two aliquots of the first ATPS composition to form the first phase solution for step (c).

In some embodiments, the extraction column is a spin column, and wherein step (e) further comprise the following steps:
(i) loading a portion of the mixed solution onto the extraction column;
(ii) centrifuging the extraction column and discarding the flow-through (also referred to as ‘supernatant’); and
(iii) repeating steps (i) and (ii) above until all of the mixed solution has been passed through the extraction column.

In some embodiments, the method further comprises the step of:
(g) subjecting said final solution to a diagnostic assay for detection and quantification of said target analyte (s).

In another aspect, provided is a method for concentrating and purifying one or more target analytes from a bulk fluid sample, comprising the steps of:
(a) dividing the bulk fluid sample containing the target analyte (s) into at least two aliquots of a sample solution;
(b) preparing at least two aliquots of a first aqueous two-phase system (ATPS) composition, wherein the first ATPS composition comprises polymers, salts, surfactants, or combinations thereof dissolved in an aqueous solution to form a first phase solution and a second phase solution;
(c) adding each aliquot of said sample solution containing the target analyte (s) to each aliquot of the first ATPS composition, such that the target analyte (s) partition to the first phase solution;
(d) collecting the first phase solutions of the at least two aliquots of the first ATPS composition, and mixing the first phase solutions with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution;
(e) collecting the third phase solution and mixing the third phase solution with a binding buffer to form a mixed solution, wherein the binding buffer comprises at least one chaotropic agent;
(f) loading the mixed solution onto an extraction column configured to selectively extract and purify the target analyte (s);
(g) eluting and collecting the target analyte (s) from the extraction column, resulting in a final solution containing the concentrated and purified target analyte (s).

In another aspect, provided is a method for concentrating and purifying one or more target analytes from a bulk fluid sample, comprising the steps of:
(a) dividing the bulk fluid sample containing the target analyte (s) into at least two aliquots of a sample solution;
(b) preparing at least two aliquots of a first aqueous two-phase system (ATPS) composition, wherein the first ATPS composition comprises polymers, salts, surfactants, or combinations thereof dissolved in an aqueous solution to form a first phase solution and a second phase solution;
(c) adding each aliquot of said sample solution containing the target analyte (s) to each aliquot of the first ATPS composition, such that the target analyte (s) partition to the first phase solution;
(d) collecting the first phase solutions of the at least two aliquots of the first ATPS composition, and mixing the first phase solutions with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution;
(e) collecting the third phase solution and mixing the third phase solution with a binding buffer to form a mixed solution, wherein the binding buffer comprises at least one chaotropic agent;
(f) loading a portion of the mixed solution onto an extraction column configured to selectively extract and purify the target analyte (s);
(g) centrifuging the extraction column and discarding the flow-through;
(h) repeating steps (f) and (g) above until all of the mixed solution has been passed through the extraction column;
(i) eluting and collecting the target analyte (s) from the extraction column; resulting in a final solution containing the concentrated and purified target analyte (s); and
(j) subjecting said final solution to a diagnostic assay for detection and quantification of said target analyte (s).

In some embodiments, the bulk fluid sample is selected from the group consisting of blood, plasma, serum, cerebrospinal fluid, urine, saliva, fecal matter, tear, sputum, nasopharyngeal mucus, vaginal discharge and penile discharge. In some embodiments, the bulk fluid sample is sample matrices that have been dissolved in a suitable preparation buffer, for example, fecal matter dissolved in a suitable volume (e.g. 500 mL) of water.

In some embodiments, the bulk fluid sample is urine.

In some embodiments, the bulk fluid sample has a volume of 40 mL or more, such as 50 mL, 60 mL, 70 mL, 80 mL, 90 mL, 100 mL, 200 mL, 300 mL, 400 mL, 500 mL or more.

In some embodiments, each aliquot of said sample solution has a volume of up to 25 ml, 26 mL, 27 mL, 28 mL, 29 mL, 30 mL, 31 mL, 32 mL, 33 mL, 34 mL, 35 mL, 36 mL, 37 mL, 38 mL, 39 mL, or 40 mL.

In some embodiments, the target analytes are selected from the group consisting of nucleic acids, a protein, an antigen, a biomolecule, a sugar moiety, a lipid, a sterol, and combinations thereof.

In some embodiments, the target analytes are DNA.

In some embodiments, the target analytes are cell-free DNA or circulating tumor DNA.

In some embodiments, the polymers are dissolved in the aqueous solution at a concentration of 4%-84% (w/w).

In some embodiments, the salts are dissolved in the aqueous solution at a concentration of 1%-55% (w/w). In some embodiments, the salts are dissolved in the aqueous solution at a concentration of 8%-55% (w/w).

In some embodiments, the surfactants are dissolved in the aqueous solution at a concentration of 0.05%-10% (w/w). In some embodiments, the surfactants are dissolved in the aqueous solution at a concentration of 0.05%-9.8% (w/w).

Another aspect provides an ATPS composition selected from the group consisting of A1, A2, A3, A4, AA1, AA2, AA3, and AA4 in Table 1a.

Another aspect provides a kit comprising a first ATPS composition selected from the group consisting of A1, A2, A3, and A4 in Table 1a; a second ATPS composition selected from the group consisting of AA1, AA2, AA3, and AA4 in Table 1a; and a binding buffer selected from the group consisting of B1, B2, and B3 in Table 1a.

In some embodiments, the kit further comprises an extraction column.

Various ATPS systems that can be used in various embodiments of the present invention include, but are not limited to, polymer-polymer, polymer-salt, polymer-surfactant, salt-surfactant, surfactant, surfactant-surfactant, or polymer-salt-surfactant.

In one embodiment, the first and/or second ATPS composition comprises a polymer. In some embodiments, polymers that may be employed include, but are not limited to, polyalkylene glycols, such as hydrophobically modified polyalkylene glycols, poly (oxyalkylene) polymers, poly (oxyalkylene) copolymers, such as hydrophobically modified poly (oxyalkylene) copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. In another embodiment, the first phase forming polymer comprises polyethylene glycol (PEG), polypropylene glycol (PPG), or dextran. In some embodiments, the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymer thereof. In some embodiments, the polymer is selected from the group consisting of dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide and copolymers thereof. In some embodiments, the polymer is selected from the group consisting of dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether and poly N-isopropylacrylamide. In some embodiments, the polymer is selected from the group consisting of polyacrylamide, polyacrylic acid and copolymers thereof. In some embodiments, the polymer is selected from the group consisting of dextran, carboxymethyl dextran, dextran sulfate, hydroxypropyl dextran and starch. In some embodiments, the polymer has an average molecular weight in the range of 200-1,000 Da, 200-35,000 Da, 425-2,000 Da, 400-35,000 Da, 980-12,000 Da, or 3, 400-5,000,000 Da. In some embodiments, the polymer comprises ethylene oxide and propylene oxide units, and the polymer has an EO:PO ratio of 90:10 to 10:90.

In one embodiment, the polymer concentration of the first and/or second ATPS composition is in the range of about 4% to about 84% by weight of the total weight of the aqueous solution (w/w). In various embodiments, the polymer solution is selected from a polymer solution that is about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 12.5% w/w, about 13% w/w, about 13.5% w/w, about 14% w/w, about 14.5% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 20.5% w/w, about 21% w/w, about 21.5% w/w, about 22% w/w, about 22.5% w/w, about 23% w/w, about 23.5% w/w, about 24% w/w, about 24.5% w/w, about 35% w/w, about 35.5% w/w, about 36% w/w, about 36.5% w/w, about 37% w/w, about 37.5% w/w, about 38% w/w, about 38.5% w/w, about 39% w/w, about 39.5% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5% w/w, about 45% w/w, about 45.5% w/w, about 46% w/w, about 46.5% w/w, about 47% w/w, about 47.5% w/w, about 48% w/w, about 48.5% w/w, about 49% w/w, about 49.5% w/w, about 50% w/w, about 50.5% w/w, about 51% w/w, about 51.5% w/w, about 52% w/w, about 52.5% w/w, about 53% w/w, about 53.5% w/w, about 54% w/w, about 54.5% w/w, about 55% w/w, about 55.5% w/w, about 56% w/w, about 56.5% w/w, about 57% w/w, about 57.5% w/w, about 58% w/w, about 58.5% w/w, about 59% w/w, about 59.5% w/w, about 60% w/w, about 60.5% w/w, about 61% w/w, about 61.5% w/w, about 62% w/w, about 62.5% w/w, about 63% w/w, about 63.5% w/w, about 64% w/w, about 64.5% w/w, about 65% w/w, about 65.5% w/w, about 66% w/w, about 66.5% w/w, about 67% w/w, about 67.5% w/w, about 68% w/w, about 68.5% w/w, about 69% w/w, about 69.5% w/w, about 70% w/w, about 70.5% w/w, about 71% w/w, about 71.5% w/w, about 72% w/w, about 72.5% w/w, about 73% w/w, about 73.5% w/w, about 74% w/w, about 74.5% w/w, about 75% w/w, about 75.5% w/w, about 76% w/w, about 76.5% w/w, about 77% w/w, about 77.5% w/w, about 78% w/w, about 78.5% w/w, about 79% w/w, about 79.5% w/w, about 80% w/w, about 80.5% w/w, about 81% w/w, about 81.5% w/w, about 82% w/w, about 82.5% w/w, about 83% w/w, about 83.5% w/w, and about 84% w/w.

In one embodiment, the first and/or second ATPS composition comprises a salt and thereby forms a salt solution. In some embodiments, the salt includes, but is not limited to, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate. In another embodiment, the salt comprises NaCl, Na₃PO₄, K₃PO₄, Na₂SO₄, potassium citrate, (NH₄)₂SO₄, sodium citrate, sodium acetate or combinations thereof. Other salts, e.g. ammonium acetate, may also be used. In another embodiment, the salt may be selected from magnesium salt, a lithium salt, a sodium salt, a potassium salt, a cesium salt, a zinc salt and an aluminum salt. In some embodiments, the salt may be selected from a bromide salt, an iodide salt, a fluoride salt, a carbonate salt, a sulfate salt, a citrate salt, a carboxylate salt, a borate salt, and a phosphate salt. In some embodiments, the salt comprises potassium phosphate. In some embodiments, the salt comprises ammonium sulfate.

In one embodiment, the total salt concentration is in the range of about 0.01% to about 90%. A skilled person in the art will understand that the amount of salt needed to form an aqueous two-phase system will be influenced by molecular weight, concentration and physical status of the polymer.

In various embodiments, the salt concentration is about 1%-55% w/w. In various embodiments, the salt concentration is about 1% w/w, about 1.5% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 12.5% w/w, about 13% w/w, about 13.5% w/w, about 14% w/w, about 14.5% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 20.5% w/w, about 21% w/w, about 21.5% w/w, about 22% w/w, about 22.5% w/w, about 23% w/w, about 23.5% w/w, about 24% w/w, about 24.5% w/w, about 35% w/w, about 35.5% w/w, about 36% w/w, about 36.5% w/w, about 37% w/w, about 37.5% w/w, about 38% w/w, about 38.5% w/w, about 39% w/w, about 39.5% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5% w/w, about 45% w/w, about 45.5% w/w, about 46% w/w, about 46.5% w/w, about 47% w/w, about 47.5% w/w, about 48% w/w, about 48.5% w/w, about 49% w/w, about 49.5% w/w, about 50% w/w, about 50.5% w/w, about 51% w/w, about 51.5% w/w, about 52% w/w, about 52.5% w/w, about 53% w/w, about 53.5% w/w, about 54% w/w, about 54.5% w/w, about 55% w/w, about 55.5% w/w, about 56% w/w, about 56.5% w/w, about 57% w/w, about 57.5% w/w, about 58% w/w, about 58.5% w/w, about 59% w/w, about 59.5% w/w, about 60% w/w, about 60.5% w/w, about 61% w/w, about 61.5% w/w, about 62% w/w, about 62.5% w/w, about 63% w/w, about 63.5% w/w, about 64% w/w, about 64.5% w/w, about 65% w/w, about 65.5% w/w, about 66% w/w, about 66.5% w/w, about 67% w/w, about 67.5% w/w, about 68% w/w, about 68.5% w/w, about 69% w/w, about 69.5% w/w, about 70% w/w, about 70.5% w/w, about 71% w/w, about 71.5% w/w, about 72% w/w, about 72.5% w/w, about 73% w/w, about 73.5% w/w, about 74% w/w, about 74.5% w/w, about 75% w/w, about 75.5% w/w, about 76% w/w, about 76.5% w/w, about 77% w/w, about 77.5% w/w, about 78% w/w, about 78.5% w/w, about 79% w/w, about 79.5% w/w, or about 80% w/w.

In one embodiment, the first and/or second ATPS composition comprises a surfactant. In some embodiments, possible surfactants that may be employed include, but are not limited to, Triton-X, Triton-114, Igepal CA-630 and Nonidet P-40, anionic surfactants, such as carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, nonionic surfactants, such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants, such as quaternary ammonium salts, amines with amide linkages, polyoxyethylene alkyl & alicyclic amines, n, n, n′, n′ tetrakis substituted ethylenediamines, 2-alkyl 1-hydroxethyl 2-imidazolines, and amphoteric surfactants, such as n-coco 3-aminopropionic acid and sodium salt thereof, n-tallow 3-iminodipropionate and disodium salt thereof, n-carboxymethyl n dimethyl n-9 octadecenyl ammonium hydroxide, n-cocoamidethyl n hydroxyethylglycine and sodium salt thereof.

In one embodiment, the surfactant concentration of the first ATPS composition is in the range of about 0.05% w/w to about 10% w/w. In various embodiments, the surfactant concentration is about 0.05% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5% w/w, about 5.1% w/w, about 5.2% w/w, about 5.3% w/w, about 5.4% w/w, about 5.5% w/w, about 5.6% w/w, about 5.7% w/w, about 5.8% w/w, about 5.9% w/w, about 6% w/w, 6.1% w/w, about 6.2% w/w, about 6.3% w/w, about 6.4% w/w, about 6.5% w/w, about 6.6% w/w, about 6.7% w/w, about 6.8% w/w, about 6.9% w/w, about 7% w/w, about 7.1% w/w, about 7.2% w/w, about 7.3% w/w, about 7.4% w/w, about 7.5% w/w, about 7.6% w/w, about 7.7% w/w, about 7.8% w/w, about 7.9% w/w, about 8% w/w, about 8.1% w/w, about 8.2% w/w, about 8.3% w/w, about 8.4% w/w, about 8.5% w/w, about 8.6% w/w, about 8.7% w/w, about 8.8% w/w, about 8.9% w/w, about 9% w/w, 9.1% w/w, about 9.2% w/w, about 9.3% w/w, about 9.4% w/w, about 9.5% w/w, about 9.6% w/w, about 9.7% w/w, about 9.8% w/w, about 9.9% w/w, or about 10% w/w.

In one embodiment, the binding buffer comprises a chaotropic agent. In some embodiments, possible chaotropic agents include, but are not limited to, n-butanol, ethanol, guanidinium chloride, guanidinium thiocyanate, lithium perchlorate, lithium acetate, magnesium chloride, phenol, 2-propanol, sodium dodecyl sulfate, thiourea, and urea.

In one embodiment, the concentration of the chaotropic agent in the binding buffer is in the range of about 0.1 M to 8 M. In various embodiments, the concentration of the chaotropic agent is about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, about 5 M, about 5.1 M, about 5.2 M, about 5.3 M, about 5.4 M, about 5.5 M, about 5.6 M, about 5.7 M, about 5.8 M, about 5.9 M, about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, or about 8 M.

In one embodiment, the possible extraction columns that may be employed include, but are not limited to, Epoch life science—EconoSpin Silica Membrane Mini Spin Column—1920-250, HiBinds RNA mini—RNACOL-02, Corbition silica spin column—PC0054, PuroSpin micro silica spin—Luna Nano USP003, Purospin nano silica spin—Lunonano USP002, Qiagen RNEasy minElute, Qiagen minElute—700384 Qiagen GMBH, and Qiagen mini.

### Embodiment 2

In some embodiments, provided is a method for concentrating and purifying one or more target analyte (s) from a bulk fluid sample, including the steps of: (a) dividing the bulk fluid sample containing the target analyte (s) into at least two aliquots of a sample solution; (b) preparing at least two first aqueous two-phase system (ATPS) compositions, wherein each first ATPS composition includes a polymer, a salt, a surfactant, or any combination thereof dissolved in an aqueous solution to form a first phase solution and a second phase solution; (c) adding each aliquot of said sample solution prepared from the bulk fluid sample containing the target analyte (s) to each first ATPS composition, such that the target analyte (s) partition to each first phase solution; (d) further processing each first phase solution to form a final phase solution; (e) mixing the final phase solution with at least one purifying composition to form a mixed solution; (f) contacting the mixed solution with a downstream purification system configured to selectively isolate the target analyte (s); and (g) collecting the target analyte (s) from the downstream purification system, resulting in a final solution containing the concentrated and purified target analyte (s).

In some embodiments, the further processing of step (d) includes collecting and combining each first phase solution to form the final phase solution.

In some embodiments, the further processing of step (d) includes the steps of (i) collecting each first phase solution; (ii) mixing each first phase solution with a second ATPS composition, wherein the second ATPS composition includes polymers, salts, surfactants, or combinations thereof dissolved in an aqueous solution to form a third phase solution and a fourth phase solution, such that the target analyte (s) partition to and concentrate in the third phase solution; (iii) collecting and combining the third phase solutions to form the final phase solution.

In some embodiments, the further processing of step (d) includes the steps of (i) collecting and combining each first phase solution to form a combined first phase solution; (ii) mixing each combined first phase solution with a second ATPS composition, wherein the second ATPS composition includes polymers, salts, surfactants, or combinations thereof dissolved in an aqueous solution to form a third phase solution and a fourth phase solution, such that the target analyte (s) partition to and concentrate in the third phase solution; (iii) collecting the third phase solution to form the final phase solution.

In some embodiments, the purifying composition is a binding buffer including at least one chaotropic agent; the downstream purification system includes a solid phase medium; and step (f) further includes the following steps: (i) contacting a portion of the mixed solution with the solid phase medium such that the target analyte (s) binds to the solid phase medium to form a solid phase extraction complex; (ii) perturbing the solid phase extraction complex and discarding the flow-through or supernatant; and (iii) optionally repeating steps (i) and (ii).

In some embodiments, the solid phase medium is a solid phase extraction column.

In some embodiments, the solid phase extraction column is a spin column.

In some embodiments, provided is a method wherein the solid phase medium is a plurality of beads.

In some embodiments, the plurality of beads is magnetic beads, silica-based beads, carboxyl beads, hydroxyl beads, amine-coated beads, or any combination thereof.

In some embodiments, provided is a method, further including the step of: (h) subjecting said final solution to a diagnostic assay for detection, quantification, characterization, or combinations thereof, of said target analyte (s).

In some embodiments, provided is a method for concentrating and purifying one or more target analytes from a bulk fluid sample, including the steps of: (a) dividing the bulk fluid sample containing the target analyte (s) into at least two aliquots of a sample solution; (b) preparing at least two aliquots of a first aqueous two-phase system (ATPS) composition, wherein the first ATPS composition includes polymers, salts, surfactants, or combinations thereof dissolved in an aqueous solution to form a first phase solution and a second phase solution; (c) adding each aliquot of said sample solution containing the target analyte (s) to each aliquot of the first ATPS composition, such that the target analyte (s) partition to the first phase solution; (d) collecting the first phase solutions of the at least two aliquots of the first ATPS composition, and mixing the first phase solutions with a second ATPS composition, wherein the second ATPS composition includes polymers, salts, surfactants, or combinations thereof dissolved in an aqueous solution to form a third phase solution and a fourth phase solution, such that the target analyte (s) partition to and concentrate in the third phase solution; (e) collecting the third phase solution and mixing the third phase solution with a binding buffer to form a mixed solution, wherein the binding buffer includes at least one chaotropic agent; (f) loading the mixed solution onto an extraction column configured to selectively extract and purify the target analyte (s); (g) eluting and collecting the target analyte (s) from the extraction column.

In some embodiments, the bulk fluid sample is selected from the group consisting of blood, plasma, serum, cerebrospinal fluid, urine, saliva, fecal matter, tear, sputum, nasopharyngeal mucus, vaginal discharge and penile discharge.

In some embodiments, the bulk fluid sample is urine.

In some embodiments, the bulk fluid sample has a volume of at least 10 mL.

In some embodiments, the bulk fluid sample has a volume of 40 mL or more.

In some embodiments, each aliquot of said sample solution has a volume of up to 40 ml.

In some embodiments, each aliquot of said sample solution has a volume of 10 to 40 mL.

In some embodiments, the target analyte (s) is selected from the group consisting of a nucleic acid, a protein, an antigen, a biomolecule, a sugar moiety, a lipid, a sterol, and any combination thereof.

In some embodiments, the target analyte (s) is DNA.

In some embodiments, the target analyte (s) is gDNA, cDNA, plasmid DNA, mitochondrial DNA, cell-free DNA (cfDNA), circulating tumor DNA (ctDNA), circulating fetal DNA, cell-free microbial DNA, micro RNA (miRNA), messenger RNA (mRNA), transfer RNA (tRNA), ribosomal RNA (rRNA), circular RNA, long non-coding RNA (lncRNA) or combinations thereof.

In some embodiments, the target analyte (s) is cell-free DNA (cfDNA) or circulating tumor DNA (ctDNA).

In some embodiments, the polymer is dissolved in an aqueous solution at a concentration of 0.5-80% (w/v).

In some embodiments, the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymers thereof. In some embodiments, the polymer is hydrophobically-modified, or silicone-modified.

In some embodiments, the polymer is dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide or copolymers thereof.

In some embodiments, the polymer is dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether or poly N-isopropylacrylamide.

In some embodiments, the polymer is a polyacrylamide, polyacrylic acid or copolymers thereof. In some embodiments, the polymer is dextran, carboxymethyl dextran, dextran sulfate, hydroxypropyl dextran or starch.

In some embodiments, the polymer has an average molecular weight in the range of 200-1,000 Da, 200-35,000 Da, 425-2,000 Da, 400-35,000 Da, 980-12,000 Da, or 3, 400-5,000,000 Da. In some embodiments, the polymer comprises ethylene oxide and propylene oxide units. In some embodiments, the polymer has an EO:PO ratio of 90:10 to 10:90.

In some embodiments, the salt is dissolved in an aqueous solution at a concentration of 0.1% to 80% (w/v).

In some embodiments, the salt includes a cation selected from the group consisting of sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium and tetrabutyl ammonium.

In some embodiments, the salt includes an anion selected from the group consisting of phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, and tris.

In some embodiments, the salt is selected from the group consisting of aluminum chloride, aluminum phosphate, aluminum carbonate, magnesium chloride, magnesium phosphate, and magnesium carbonate.

In some embodiments, the salt is selected from the group consisting of NaCl, KCl, NH₄Cl, Na₃PO₄, K₃PO₄, Na₂SO₄, K₂HPO₄, KH₂PO₄, Na₂HPO₄, NaH₂PO₄, (NH₄)₃PO₄, (NH₄)₂HPO₄, NH₄H₂PO₄, potassium citrate, (NH₄)₂SO₄, sodium citrate, sodium acetate, magnesium acetate, sodium oxalate, sodium borate, and ammonium acetate.

In some embodiments, the salt is selected from the group consisting of (NH₄)₃PO₄, sodium formate, ammonium formate, K₂CO₃, KHCO₃, Na₂CO₃, NaHCO₃, MgSO₄, MgCO₃, CaCO₃, CsOH, Cs₂CO₃, Ba(OH)₂, and BaCO₃.

In some embodiments, the salt is selected from the group consisting of NH₄Cl, NH₄ OH, tetramethyl ammonium chloride, tetrabutyl ammonium chloride, tetramethyl ammonium hydroxide, and tetrabutyl ammonium hydroxide.

In some embodiments, the surfactant is dissolved in an aqueous solution at a concentration of 0.05%-10% (w/w).

In some embodiments, the surfactant is selected from the group consisting of anionic surfactant, nonionic surfactant, cationic surfactant, and amphoteric surfactant; and wherein the anionic surfactant is carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, or sodium N-lauroyl sarcosinate (NLS); the nonionic surfactant is ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, or polyoxyethylene fatty acid amides; the cationic surfactant is quaternary ammonium salts, amines with amide linkages, polyoxyethylene alkyl amines, polyoxyethylene alicyclic amines' n′n, n′, n′tetrakis substituted ethylenediamines, or 2-alkyl 1-hydroxethyl 2-imidazolines; and the amphoteric surfactant is n-coco 3-aminopropionic acid or sodium salt thereof, n-tallow 3-iminodipropionate or disodium salt thereof, n-carboxymethyl n dimethyl n-9 octadecenyl ammonium hydroxide, or n-cocoamidethyl n hydroxyethylglycine or sodium salt thereof.

In some embodiments, the surfactant is Triton X-100, Triton X-114, Triton X-45, Tween 20, Igepal CA630, Brij 58, Brij O10, Brij L23, Pluronic L-61, Pluronic F-127, UCON, sodium dodecyl sulfate, sodium cholate, sodium deoxycholate, sodium N-lauroyl sarcosinate (NLS), hexadecyltrimethlammonium bromide, or span 80.

In some embodiments, said binding buffer is a chaotropic agent including an anion selected from the group consisting of thiocyanate, isothiocyanate, perchlorate, acetate, trichloroacetate, trifluoroacetate, chloride, and iodide.

In some embodiments, said binding buffer is a chaotropic agent selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

In some embodiments, said binding buffer is a chaotropic agent selected from the group consisting of guanidinium hydrochloride, magnesium chloride, and guanidinium thiocyanate.

In some embodiments, the binding buffer includes a chaotropic agent at a concentration of 2-7M.

In some embodiments, the first ATPS composition includes said polymer at a concentration of 5-80% (w/v), said salt at a concentration of 0.1-80% (w/v), and said surfactant at a concentration of 0-10% (w/v); the volume ratio of the first phase solution to the second phase solution is A:B, and wherein A is 1 and B is 0.9 to 13.

In some embodiments, the second ATPS composition includes said polymer at a concentration of 0.5-30% (w/v), said salt at a concentration of 0.1-10% (w/v), and said surfactant at a concentration of 0-10% (w/v); the volume ratio of the third phase solution to the fourth phase solution is C:D; and wherein C is 1 and D is 1 to 24.

In some embodiments, the first ATPS composition includes 5-80% polymer (w/v) and 0.1-80% salt (w/v); and the second ATPS composition includes 0.5-30% polymer (w/v) and 5-60% salt (w/v).

In some embodiments, the first ATPS composition includes 5-60% polymer (w/v) and 0.5-50% salt (w/v); and the second ATPS composition includes 0.5-30% polymer (w/v) and 5-60% salt (w/v).

In some embodiments, the first ATPS composition includes 12-50% polymer (w/v) and 0.1-20% salt (w/v); and the second ATPS composition includes 0.5-30% polymer (w/v) and 5-60% salt (w/v).

In some embodiments, the first ATPS composition further includes 0.5-2 mM ethylenediaminetetraacetic acid (EDTA), and 0.01-10% surfactant; and the second ATPS composition further includes 0.5-2 mM EDTA.

In some embodiments, the volume ratio between the first phase solution and the second phase solution of the first ATPS composition is A:B. wherein A is 0.1 to 19 and B is 1.

In some embodiments, A is 0.9 to 13 and B is 1.

In some embodiments, A:B is 13:1, 6:1, or 0.9:1.

In some embodiments, the volume ratio between the third phase solution and the fourth phase solution of the second ATPS composition is C:D. wherein C is 1 and D is greater than or equal to 4.

In some embodiments, D is 4-100.

In some embodiments, D is 24.

In some embodiments, A is 5-15; B 1; C is 1; and D is 20-100.

In some embodiments, provided is an ATPS composition selected from the group consisting of A1, A2, A3, A4, AA1, AA2, AA3, and AA4.

In some embodiments, provided is a method 1, analyzing the final solution, and treating the patient with a cancer therapeutic if the target analyte indicates that the patient has bladder cancer or is at risk of developing bladder cancer.

In some embodiments, provided is a kit including a first ATPS composition selected from the group consisting of A1, A2, A3, and A4; a second ATPS composition selected from the group consisting of AA1, AA2, AA3, and AA4; and a binding buffer selected from the group consisting of B1, B2, and B3.

In some embodiments, the kit further includes an extraction column.

In some embodiments, the polymer is at a concentration of 0.5-80% (w/v) of the first ATPS and/or the second ATPS. In some embodiments, the polymer is at a concentration of 0.5-30% (w/v) of the first ATPS and/or the second ATPS. In some embodiments, the polymer is at a concentration of 5-60% (w/v) of the first ATPS and/or the second ATPS. In some embodiments, the polymer is at a concentration of 12-50% (w/v) of the first ATPS and/or the second ATPS.

In some embodiments, the salt is at a concentration of 0.1%-80% (w/v) of the first ATPS and/or the second ATPS. In some embodiments, the salt is at a concentration of 5%-60% (w/v) of the first ATPS and/or the second ATPS. In some embodiments, the salt is at a concentration of 0.1%-50% (w/v) of the first ATPS and/or the second ATPS. In some embodiments, the salt is at a concentration of 0.1%-20% (w/v) of the first ATPS and/or the second ATPS. In some embodiments, the salt is at a concentration of 0.01%-30% (w/v). In some embodiments, the salt is at a concentration of 0.01%-10% (w/v) of the first ATPS and/or the second ATPS.

In some embodiments, the surfactant is at a concentration of 0.1-50% (w/v) of the first ATPS and/or the second ATPS. In some embodiments, the surfactant is at a concentration of 0.01%-10% (w/v) of the first ATPS and/or the second ATPS.

In some embodiments, the first ATPS composition is polymer-salt based, comprising at least one polymer at a concentration of 5-80% (w/v) and at least one salt at a concentration of 0.1-80% (w/v). In some embodiments, the first ATPS composition comprises at least one polymer at a concentration of 5-60% (w/v) and at least one salt at a concentration of 0.5-50% (w/v). In some embodiments, the first ATPS composition comprises at least one polymer at a concentration of 12-50% (w/v) and at least one salt a concentration of 0.1-20% (w/v). In some embodiments, the first ATPS composition further comprises at least one surfactant at a concentration of 0.01%-10% (w/v).

In some embodiments, the second ATPS composition comprises at least one polymer at a concentration of 0.5-30% (w/v) and at least one salt at a concentration of 5-60% (w/v). In some embodiments, the second ATPS composition comprises at least one polymer at a concentration of 1-6% (w/v) and at least one salt at a concentration of 10-50% (w/v). In some embodiments, the second ATPS composition further comprises at least one surfactant at a concentration of 0.01%-10% (w/v).

In some embodiments, the first ATPS composition is polymer-salt based, comprising at least one polymer at a concentration of 0.5-30% (w/v) and at least one salt at a concentration of 5-60% (w/v). In some embodiments, the first ATPS composition comprises at least one polymer at a concentration of 1-6% (w/v) and at least one salt at a concentration of 10-50% (w/v). In some embodiments, the first ATPS composition further comprises at least one surfactant at a concentration of 0.01%-10% (w/v).

In some embodiments, the second ATPS composition comprises at least one polymer at a concentration of 5-80% (w/v) and at least one salt at a concentration of 0.1-80% (w/v). In some embodiments, the second ATPS composition comprises at least one polymer at a concentration of 5-60% (w/v) and at least one salt at a concentration of 0.5-50% (w/v). In some embodiments, the second ATPS composition comprises at least one polymer at a concentration of 12-50% (w/v) and at least one salt at a concentration of 0.1-20% (w/v). In some embodiments, the second ATPS composition further comprises at least one surfactant at a concentration of 0.01%-10% (w/v).

In some embodiments, the first ATPS composition is polymer-polymer based, comprising at least one polymer at a concentration of 0.2-50% (w/v). In some embodiments, the first ATPS composition further comprises at least one salt at a concentration of 0.01%-10% (w/v). In some embodiments, the first ATPS composition further comprises at least one surfactant at a concentration of 0.01%-10% (w/v).

In some embodiments, the first ATPS composition is surfactant based, comprising at least one surfactant at a concentration of 0.1-50% (w/v). In some embodiments, the first ATPS composition further comprises at least one salt at a concentration of 0.01%-30% (w/v).

Although the description refers to particular embodiments, the disclosure should not be construed as limited to the embodiments set forth herein.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

The following equipment are used for the methods in Examples 1 and 2 below:
1. Centrifuge (e.g., for 15 and 50 mL conical tubes).
2. Benchtop microcentrifuge (e.g., for 1 and 2 mL tubes).
3. Pipettes and pipette tips (e.g., of 20 μL, 200 μL and 1000 μL capacity pipettes).
4. Pipette aid and serological pipette tips (e.g., of 5 mL, 10 mL, and 50 mL).
5. Water bath (e.g., set at 37° C.).

### Example 1: Concentrating and Isolating a Target Analyte from a 40 mL Sample

Below is an example method of how to concentrate and isolate a target analyte from a biological sample that has a volume of at least 40 mL. In this example, samples are prepared following the steps below:
1. 40 mL of a biological sample is mixed with at least one lysing reagent (optional) using methods known to one of skill in the art to form one or more sample lysates.
2. A portion of the sample lysate (22.6 mL) is transferred into a first tube containing the first ATPS composition (ATPS #1) to form an ATPS #1 solution. The remaining sample lysate is poured into a second tube also containing ATPS #1 to form an ATPS #1 solution.
3. Both tubes containing the ATPS #1 solutions are vortexed thoroughly until homogenous and then each centrifuged for 6 minutes at 2300 RCF.
4. The bottom phases from the two ATPS #1 solution (e.g., around 3.5-5 mL of volume) are transferred (e.g., using a 10 mL serological pipette) into a tube containing a second ATPS composition (ATPS #2) to form an ATPS #2 solution. The ATPS #2 solution is vortexed thoroughly until homogenous, and then centrifuged for 6 minutes at 2300 RCF.
5. All of the top phases (around 400-600 uL) of the ATPS #2 solutions are transferred into a 5 mL microcentrifuge tube. ˜2 mL of binding buffer is added to the microcentrifuge tube containing the ATPS #2 top phase, and the tube is vortexed briefly.
6. 800 μL of the ATPS #2 top phase is transferred to a spin column and centrifuged for 30 sec at 12,000 rcf. The flow-through (also referred as ‘supernatant’) is discarded. This spin column step (step 6) is repeated until all samples have passed through the spin column.
7. Wash buffer is added (500 uL) to the spin column containing the mixture and the spin column containing the mixture is centrifuged for 30 sec at 12,000 rcf. The flow-through is discarded.
8. The spin column containing the mixture is centrifuged for 2 min at 16,000 rcf to remove any excess wash buffer.
9. The spin column containing the mixture is placed in a new 1.5 mL collection tube. 1×TE buffer (20-100 uL) is pipetted into the center of the spin column membrane. The spin column containing the mixture is incubated for 3 min and centrifuged at 1 min at 12,000 rcf to elute a sample solution containing the concentrated target analyte. The sample solution is stored in a freezer at −20° C. or below for optional further processing.

The above example procedure is just one example, and alternate methods and conditions may also be used.

For example, in step 2, the 40 mL sample after being subjected to the lysing agent is roughly split into two portions. However, bulk fluid samples can be divided up in a number of different permutations.

Specific examples of ATPS #1, ATPS #2, and binding buffers that can be used in the above protocol are shown in Table 1a below.

### Example 2: Concentrating and Isolating a Target Analyte from a 160 mL Sample

In this example, a larger bulk volume sample of around 160 mL is prepared following the steps below:
1. The 160 mL sample is divided into four separate 40 mL portions. For each 40 mL volume of sample input, steps (1) through (7) from the above Example 1 are performed.
2. The top phases of each ATPS #2 (about 400 uL-600 uL) is transferred into a 15 mL microcentrifuge tube. This extraction step may be done with a pipette, such a P200 pipette set to 200 uL for the first extraction.
3. For each 40 mL of starting sample, binding buffer (2 mL) is added to the tube containing the ATPS #2 top phases (ie. 160 mL sample scale up would require 4× ATPS #2 and 8 mL binding buffer). The tube is vortexed briefly.
4. 800 μL of the mixture (of starting sample and binding buffer) is transferred to the spin column.
5. The mixture is centrifuged for 30 sec at 12,000 rcf.
6. The flow-through (also referred as ‘supernatant’) is discarded. Steps 4-6 are repeated for the remaining sample until the entire mixture has passed through the spin columns. (For example, with a 800 uL spin column capacity, a 160 mL starting sample input volume required approximately 12 cycles)
7. Wash buffer is added to the spin column containing the mixture (500 uL).
8. The spin column containing the mixture is centrifuged for 30 sec at 12,000 rcf.
9. The flow-through is discarded.
10. The spin column containing the mixture is centrifuged for 2 min at 16,000 rcf to remove any excess Wash buffer.
11. The spin column containing the mixture is placed in a new 1.5 mL collection tube.
12. 1×TE buffer (20-100 uL) is pipetted into the center of the spin column membrane.
13. The spin column containing the mixture is incubate for 3 min and centrifuged at 1 min at 12,000 rcf to elute a sample solution containing the concentrated target analyte.
14. The sample solution is stored in a freezer at −20° C. or below for optional further processing.

### Example 3: Evaluation of the Performance of the Disclosed Methods

Performance of the presently disclosed methods and kits below can be evaluated following the steps below:
1. Several high-volume extraction kit components are prepared by varying the following components:
a. ATPS #1
i. Polymer
ii. Salt
iii. Surfactant
b. ATPS #2
i. Polymer
ii. Salt
c. Extraction column
d. Binding Buffer
i. Chaotropic agent
2. Sample solutions are made to evaluate and spike in known quantities of DNA target.
3. Extractions are made using variations of high-volume extraction kits prepared in Step 1 above as well as industry standard extraction kits using their specified procedures.
4. Target DNA are quantified using standard qPCR or ddPCR procedures

TABLE 1a
List of Example Compositions
Specific examples of ATPS #1, ATPS #2, and binding buffers are shown below.

| | | | | | Chaotropic |
|---|---|---|---|---|---|
| Example | Reagent | Polymer | Salt | Surfactant | Agent |
| A1 | ATPS #1 | Polyvinyl alcohol | Sodium Sulfate | None | None |
| | | 55-63% v/v | 12-15% w/v | | |
| A2 | ATPS #1 | Polyethylene | Phosphate Salt | Nonionic | None |
| | | oxide (POE) 65- | 18-24% w/v | surfactant | |
| | | 80% v/v | | 0.05-0.4% | |
| | | | | v/v | |
| A3 | ATPS #1 | PPG 78-84% v/v | Potassium | Igepal 0.5- | None |
| | | | citrate | 1.8% v/v | |
| | | | 19-23% w/v | | |
| A4 | ATPS #1 | Dextran 42-57% | Magnesium Salt | Anionic | None |
| | | w/v | 8-12% w/v | Surfactant | |
| | | | | 2-5% v/v | |
| AA1 | ATPS #2 | Polyvinyl alcohol | Sodium Sulfate | None | None |
| | | 12-19% v/v | 32-55% w/v | | |
| AA2 | ATPS #2 | PPG 4-20% v/v | Potassium | Igepal 4.5- | None |
| | | | citrate | 9.8% v/v | |
| | | | 29-43% w/v | | |
| AA3 | ATPS #2 | Dextran 15-28% | Magnesium Salt | Anionic | None |
| | | w/v | 20-31% w/v | Surfactant | |
| | | | | 2-5% v/v | |
| AA4 | ATPS #2 | POE 8-14% v/v | Phosphate Salt | None | None |
| | | | 37-52% w/v | | |
| B1 | Binding | None | None | None | 2.5-6M |
| | Buffer | | | | Guanidinium |
| | | | | | Chloride |
| B2 | Binding | None | None | None | 3-8M |
| | Buffer | | | | Magnesium |
| | | | | | Chloride |
| B3 | Binding | None | None | None | 4-7M |
| | Buffer | | | | Guanidinium |
| | | | | | Thiocyanate |

Sample solutions spiked with known quantities of DNA were processed according to the method described in Example 2 using different combinations of ATPS compositions and binding buffers as shown in Table 1a.

### Results

The methods of the present disclosure were found to be effective at isolating and concentrating target DNA from bulk fluid sample.

The above examples are presented for illustrative purposes only and are not intended to be an exhaustive list of all possible embodiments of the invention. Other example embodiments are discussed herein.

### Example 4a: Urine Extraction Using Spin Column with and without Prior ATPS Steps

In this example, DNA recovery efficiencies from large volume of urine using spin column (i) with prior phase separation using ATPS systems (also referred to as “ATPS steps”) in accordance with the method of the present disclosure; and (ii) without prior ATPS steps are compared.

### Urine Lysis

Urine samples were collected from 4 different donors. The samples from each donor were aliquoted into tubes of 40 mL per tube and divided into 3 sets, with each set containing 1 sample from each donor.

All 3 sets of urine samples were pre-treated with 200 μL of 0.1M EDTA per 10 mL urine sample, vortexed thoroughly and centrifuged at 3000 rcf for 10 minutes. The supernatant was transferred to a new tube while the pellet was discarded.

Unwanted protein and cells present in pre-treated urine samples were lysed by adding 5.2 mL suitable lysis buffer to 40 mL of sample per donor. 100 fg of 145 bp double stranded DNA (dsDNA) and 100 ng of 1 kb+DNA ladder was spiked into the above samples. The samples were then vortexed thoroughly until homogenous then left in a pre-heated 37° C. water bath to incubate for 15 minutes.

### Two Phase System

Two different aqueous two-phase systems (ATPS) (also referred to as “dual ATPS system” or “sequential ATPS” in some embodiments) were prepared to extract cell-free DNA (cfDNA) from urine samples. The first ATPS (polymer, salts and/or surfactant) was used for initial extraction of urine sample where the intended cfDNA partitions strongly to the bottom salt-rich phase. The bottom phase from the first ATPS was then extracted and added to the second ATPS (polymer, salts and/or surfactant), which was used to concentrate the target cfDNA into a small volume (400 uL-600 uL) to allow for user-friendly downstream processing.

The first ATPS consists of 31-35% (w/v) polymer, 6-9% (w/v) salt, 1.0-1.5 mM EDTA, 0.05-0.35% (v/v) surfactant, with 22600 μL of lysed urine sample.

The second ATPS consists of 3-11% (w/v) polymer, 18-28% (w/v) salt, 1.0-1.5 mM EDTA, with 3.5 mL-5 mL of first ATPS bottom phase.

For urine sample set 1, pre-treated urine samples from each donor were split in half (22.6 mL) then added to the 2 first ATPS tubes to perform phase separation in parallel (also referred to as “parallel ATPS”). The first ATPS were vortexed thoroughly then centrifuged at 2300 rcf for 6 minutes. The salt-rich bottom phase from two first ATPS (same donor) were then extracted, recombined, and added to one tube of second ATPS, which was vortexed thoroughly and centrifuged to allow to phase separate. The polymer rich top phase of the second ATPS system was extracted and put into a new tube.

Urine sample set 2 was pre-treated and processed but did not go through the dual ATPS system for concentration and purification.

### Purification of DNA

The target cfDNA in the urine sample partitioned to the polymer-rich top phase in the second ATPS and was concentrated down to 400 uL-600 uL. The top phase was isolated for further processing.

A 3-7M solution of guanidinium was used as a binding buffer. 2 mL of the binding buffer was added to (˜400 uL-600 uL) of the extracted second ATPS polymer-rich top phase from urine set 1 and vortexed thoroughly. Urine sample set 2 (40 mL), which did not go through the ATPS purification and concentration, was mixed with 2 mL of the binding buffer and vortexed thoroughly. Each urine sample was then added to an EconoSpin column for DNA attached to the QIAvac 24 Plus vacuum manifold with the appropriate extenders (3 mL and 20 mL). A pressure of 900 mbar was applied to the vacuum manifold and the sample lysate was allowed to flow through the spin column. The target cfDNA was bound to the spin column and retained while the sample lysate flowed through was discarded by the vacuum manifold. After all possible sample lysates had flowed through the spin column, the extenders were removed and discarded. The spin columns were removed from the manifold and inserted into 2 mL waste tubes. 500 μL of RPE wash buffer (80% v/v EtOH, 0.1M sodium chloride, 0.01M Tris-HCl) were added to the spin columns and centrifuged at 12000 rcf for 30 seconds. The flow through was discarded and the spin column was further centrifuged at 16000 rcf for 2 minutes to remove any excess RPE wash buffer. The spin columns were then placed in new 1.5 mL centrifuge tubes where 80 μL of elution buffer (0.01M Tris-HCl, 1 mM EDTA) were transferred directly onto the silica membrane and allowed to incubate at room temperature for 3 minutes. The spin columns were centrifuged at 12000 rcf for 1 minute to elute the target cfDNA into 1.5 mL centrifuge tube.

### Detection of DNA

Recovery of DNA spiked into the extracted samples (145 bp and 2000 bp DNA) were quantified by qPCR using the Quant Studio 5. qPCR master mix was prepared as follows per reaction, 5 μL of TaqMan Fast Advanced Master Mix (Applied Biosystems, Ref: 4444557), 0.5 uL of 20× custom pre-mixed custom oligo PSI-145 FAM Dental, 0.4 uL of Universal Spike II Primer (TATAA, DS25SII), 0.2 uL Universal Spike II Probe (TATAA, DSSII) 1.9 uL of Ultra-Pure H2O. The results were presented as average CT values. A lower average CT value indicates a higher amount of the target DNA in the extracted samples, and a higher CT value indicates a lower amount of the target DNA in the extracted samples.

### Results

### Column Flow Speeds

The different urine sample sets flowed through the silica membrane column at different speeds despite having the same level of vacuum suction. The amount of sample lysate passed through the column and the time to pass through the column of different urine sample sets using different purification steps are summarized in Table 2a below.

TABLE 2a
Summary of results of different purification steps

| | | Binding Buffer | Amount of sample | Time to pass |
|---|---|---|---|---|
| | | (3-7M | lysate passed | through |
| | ATPS treatment | guanidinium) | through column | column |
| Sample #1 | Dual ATPS (split into | 2 mL | 100% | 1 minute |
| | two first ATPS and | | | |
| | recombined into one | | | |
| | second ATPS) | | | |
| Sample #2 | No | 2 mL | 25% | 1 hour |

### Recovery of DNA

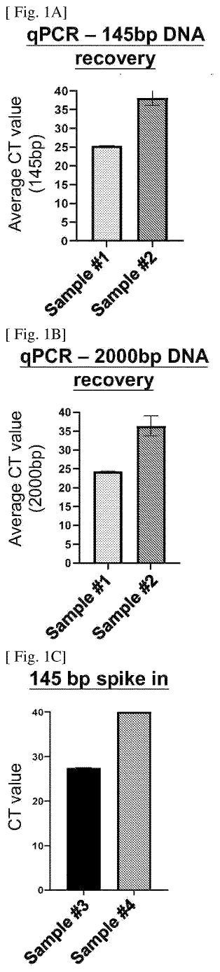

Now referring to FIGS. 1A-1B and Table 2b, both the average CT values of 145 bp DNA (FIG. 1A) and 2000 bp DNA (FIG. 1B) recovered from urine using spin column with prior ATPS steps (Sample #1) and without prior ATPS steps (Sample #2) are shown. For 145 bp DNA, Urine sample set 1 (Sample #1) had high recovery (average CT value of 25.36), yet poor recovery (average CT value of 38.15) is observed from urine Sample #2 (as shown in Table 2b). Similar results can be observed for 200 bp DNA recovery. Referring to Table 2a, in urine Sample #1, all lysate sample flowed through the spin column within 1 minute. In urine Sample #2, only 25% of the sample lysate had passed through the spin column in the first 60 minutes. Without parallel ATPS, increased flow time (1 hour) was needed in order for unprocessed lysates to pass through the column completely. As shown by the results, concentrating the urine lysates by parallel ATPS prior to spin column extraction has enhanced the efficiency of the downstream processing and significantly improved the recovery of 145 bp and 2000 bp DNA.

TABLE 2b
qPCR results of 145 bp and 2000 bp DNA oligos recovery in
urine with or without two-step ATPS using spin column.

| | 145 bp spike in | | 2000 bp spike in | |
|---|---|---|---|---|
| | Sample #1 | Sample #2 | Sample #1 | Sample #2 |
| CT value | 25.282 | 35.341 | 24.099 | 33.675 |
| | 25.419 | 40.000 | 24.427 | 40.000 |
| | 25.299 | 39.481 | 24.246 | 36.619 |
| | 25.427 | 37.780 | 24.479 | 35.390 |
| Average ± SD | 25.36 ± 0.08 | 38.15 ± 2.10 | 24.31 ± 0.17 | 36.42 ± 2.67 |

In addition, adding a parallel ATPS step in the large sample volume workflow such as urine extraction provides the following benefits when compared with non-ATPS processed samples:

### Significant Reduction in Reagent Consumption

As shown in the example herein, adding a parallel ATPS step significantly reduced the binding buffer amount needed for unprocessed lysates, for example, from 40 mL to only 2 mL. This is due to the small top polymer-rich phase produced by the second ATPS. The magnitude of reduced reagent consumption becomes even more apparent when sample input volume is increased, as a larger input volume would exponentially require more binding buffer, while ATPS processing can be modified to keep the top phase volume constant.

### Significant Decrease in Column Flow Through Time

As shown in the example herein, adding a parallel ATPS step also significantly and surprisingly reduced the column flow through time from 1 hour to 1 minute.

### Simplified Laboratory Setup

Further, as can be seen from the examples shown herein, with the much smaller sample lysate volume, custom large volume extenders would not be required when ATPS sample lysate concentration was applied. A special vacuum manifold would not be needed, and usage of centrifuges for sample lysate pass through can be achieved.

Further examples demonstrating these surprising efficiencies of a system combined with one or more ATPS extraction workflows for the extraction and purification of target analytes from large volume samples are discussed below.

### Example 4b: Urine Extraction Using Magnetic Beads with and without Prior ATPS Steps

The experiment discussed in Example 4a was repeated except the purification step was performed using magnetic beads as the solid phase.

Two urine sample sets were prepared, pre-treated and lysed according to the steps discussed in Example 4a. The urine sample sets in this example (sample #3 and sample

TABLE 2c
Summary of test conditions for urine extraction using magnetic beads

| Sample | Condition |
|---|---|
| Sample #3 | Dual ATPS (according to Example 4a; split into two first ATPS and |
| | recombined into one second ATPS), followed by magnetic beads with 2 mL |
| | binding buffer |
| Sample #4 | Magnetic beads with 2 mL binding buffer |

### Purification of DNA

The target cfDNA in the urine sample partitioned to the polymer-rich top phase in the second ATPS and been concentrated down to 400 uL-600 uL. The top phase was isolated for further processing.

3-7M guanidinium was used as a binding buffer. 2 mL of binding buffer was added respectively to the extracted top phase of Sample #1 and lysed Sample #2 which did not go through ATPS. 24 μL of magnetic bead was added into each tube. The mixture was then incubated on rotator for 5 minutes to prevent sediment of bead. The tube was then briefly spined down and placed on a magnetic rack for 2 minutes to immobilize bead at tube wall. Supernatant was discarded without disturbing the bead. 2 mL of binding buffer was added into each tube and tubes were rotated slowly on magnetic stand for 7200 in total. The supernatant was again pipetted and discarded. 800 μL of washing buffer (70% ethanol, 0.001 M EDTA, 0.01 M Tris-HCl) was added to the sample, and the tube was rotated on the rack for 7200 in total. The supernatant was discarded. The washing steps were performed twice. To enhance drying effectiveness the tubes were briefly spined down using bench-top microcentrifuge with the hinge facing outwards to collect any remaining washing buffer. The bead was then dried for 7 minutes on magnetic stand with cap opened. The bead complex was resuspended in 80 μL of Elution buffer (0.01 M Tris-HCl, 0.001 M EDTA) by continuous pipette mixing, followed by mild vortex. The tube was then placed on the magnetic rack for 1 minute. The supernatant was collected carefully into a DNA lo-bind tube (purchased from Eppendorf, catalogue #0030108035) without disturbing the magnetic beads for detection.

### Detection of DNA

The steps to perform the detection of DNA for each sample are the same or similar to those as discussed with respect to Example 4a above. For the sake of brevity and simplicity of the present disclosure, the discussion of the detection steps is not reproduced here. The results were presented as average CT values.

### Results

### Recovery of DNA

Now referring to FIGS. 1C-1D and Table 2d, both the average CT values of 145 bp DNA (FIG. 1C) and 2000 bp DNA (FIG. 1D) recovered from urine using magnetic beads with prior ATPS steps (Sample #3) and without prior ATPS steps (Sample #4) are shown. A high recovery of both the 145 bp DNA (average CT value of 27.47) and 2000 bp DNA (average CT value of 27.09) from urine Sample #3 are shown, while no detectable target DNA recovery is observed from urine Sample #4 (shown in Table 2d). The results show that under the same condition, the recovery of DNA from large volume samples such as urine was significantly improved by incorporating parallel ATPS steps prior to magnetic beads extraction.

TABLE 2d
qPCR results of 145 bp and 2000 bp DNA oligos recovery in
urine with or without two-step ATPS using magnetic beads.

| | 145 bp spike in | | 2000 bp spike in | |
|---|---|---|---|---|
| | Sample #3 | Sample #4 | Sample #3 | Sample #4 |
| CT value | 27.384 | 40.000 | 27.007 | 40.000 |
| | 27.530 | 40.000 | 27.137 | 40.000 |
| | 27.497 | 40.000 | 27.120 | 40.000 |
| Average ± SD | 27.47 ± 0.08 | 40 ± 0 | 27.09 ± 0.07 | 40 ± 0 |

### Example 5: Urine Extraction by Splitting Sample Matrix into 2× First ATPS

In this example, urine samples were split into several first ATPS and/or second ATPS (i.e. parallel ATPS), and the DNA recovery thereof was compared with urine samples processed by one single first and second ATPS.

### Urine Lysis

In this example, urine samples were pre-treated with 200 μL of 0.1 M EDTA per 10 mL urine sample, vortexed thoroughly and centrifuged at 3000 rcf for 10 minutes. The supernatant was transferred to a new tube while the pellet was discarded. To digest unwanted protein and cells present in pre-treated urine samples, 600 μL of Proteinase K (28.57 mg/mL) and 2 mL of a suitable lysis buffer were added to 20 mL of sample. 100 fg of 145 bp dsDNA and 100 ng of 1 kb+DNA ladder was spiked into the above sample. The samples were then vortexed thoroughly till homogenous then left in a pre-heated 37° C. water bath to incubate for 15 minutes.

### Two Phase System

In this example, the extraction procedure involves two sequential aqueous two-phase systems (ATPS) to isolate, purify and concentrate DNA from a urine sample. In the first ATPS, DNA partitions to the bottom phase and proteins partition to the top phase. The bottom phase, which amounts to around 5 mL, was carefully extracted and transferred to the second ATPS. In the second ATPS, DNA partitions strongly to the top phase, which amounts to around 500 uL, effectively concentrating 20 mL of sample matrix into 500 μL of target-rich phase containing target cfDNA.

Urine samples from 3 donors were split into 3 separate groups (Groups 1-3). For Group 1, 22.6 mL of urine sample were split into half and added to 2 separate first ATPS (2× first ATPS). The top phases from these first ATPS were added to 2 separate second ATPS (2× second ATPS). For Group 2, 22.6 mL of urine sample were similarly split into half and added to 2 separate first ATPS (2× first ATPS), however, the top phases from these first ATPS were combined and added to 1 single larger second ATPS (1× second ATPS). For Group 3, 22.6 mL of urine sample was added directly to 1 single large first ATPS (1× first ATPS), then the top phase was extracted into 1 single large second ATPS (1× second ATPS). To ensure an equal comparison, both first and second ATPS were scaled accordingly to ensure compositions of salt and polymer were identical between conditions. The extraction conditions of the first and second ATPS used in this experiment are summarized in Table 3. Table 4 shows the example polymer and salts combinations in the first and second ATPS compositions. The concentration of polymer and salts and/or the volume of the first and second ATPS compositions were adjusted accordingly depending on the volumes of lysates used (11.3 mL, 11.3 mL and 22.6 mL in Groups 1-3 respectively), accordingly to Table 3.

TABLE 3
Overall summary of workflow between different ATPS conditions.

| | | Scale | | Scale |
|---|---|---|---|---|
| Group | First ATPS | factor | Second ATPS | factor |
| 1 | 2x first ATPS | 0.5x | 2x second ATPS | 0.5x |
| | (each containing | | (each containing 2.5 mL first | |
| | 11.3 mL lysate) | | ATPS bottom phase) | |
| 2 | 2x first ATPS | 0.5x | 1x second ATPS | 1x |
| | (each containing | | (containing 5 mL first ATPS | |
| | 11.3 mL lysate) | | bottom phase) | |
| 3 | 1x first ATPS | 1x | 1x second ATPS | 1x |
| | (containing 22.6 mL | | (containing 5 mL first ATPS | |
| | lysate) | | bottom phase) | |

TABLE 4
Concentrations of polymer (s) and salt (s)
in first and second ATPS respectively.

| First ATPS | Second ATPS |
|---|---|
| 20-40% PAG, 0.1-20% phosphates | 3-20% PAG, 0.1-20% phosphates |

All concentrations are in w/v ratio.

### Purification of DNA

The purification of DNA was done by spin column extraction. The top phase from the second ATPS was transferred to a tube containing 1 mL of binding buffer (3-7M guanidinium) and mixed thoroughly, 800 μL of the solution was transferred to a spin column (EconoSpin) and was centrifuged for 30 seconds at 12,000 rcf. The flow-through was discarded. The process was repeated until all sample has been passed through the spin column. To the spin column was added 500 μL of washing buffer (80% ethanol v/v, 0.1 M NaCl, 0.01 M Tris-HCl), and was centrifuged for 30 seconds at 12,000 rcf. The spin column was then centrifuged for 2 minutes at 16,000 rcf to dry. 40 μL of elution buffer (0.01 M Tris-HCl, 0.001 M EDTA) was added to the spin column membrane. The spin column was incubated at room temperature for 3 minutes. The elution was collected to a collection tube by centrifuge for 1 minute at 12,000 rcf for detection.

### Detection of DNA

The steps to perform the detection of DNA for each sample are the same or similar to those as discussed with respect to Example 4a above. For the sake of brevity and simplicity of the present disclosure, the discussion of the detection steps is not reproduced here. The results are presented as average CT values.

### Results

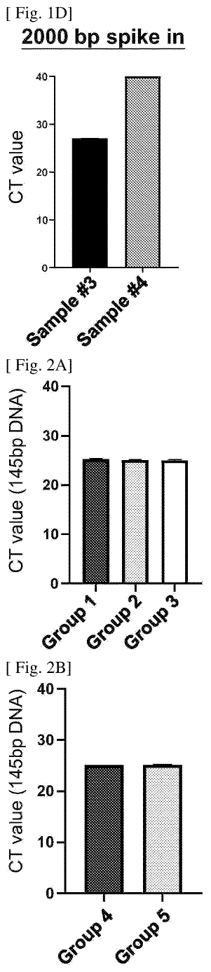

FIG. 2A shows the Ct values obtained from qPCR of 145 bp dsDNA recovered under different extraction conditions (Groups 1-3) according to Table 3. The results are also summarized in Table 5 below.

TABLE 5
Average Ct values from qPCR of 145 bp dsDNA recovered.

| | Group 1 | | | Group 2 | | | Group 3 | | |
|---|---|---|---|---|---|---|---|---|---|
| | Donor | Donor | Donor | Donor | Donor | Donor | Donor | Donor | Donor |
| | 1 | 2 | 3 | 1 | 2 | 3 | 1 | 2 | 3 |
| CT value | 25.12 | 25.23 | 25.33 | 25.02 | 24.98 | 25.16 | 25.16 | 25.02 | 24.80 |
| (145 bp) | 25.28 | 25.33 | 25.38 | 24.91 | 25.24 | 25.22 | 25.04 | 25.11 | 25.11 |
| Average ± | 25.28 ± 0.09 | | | 25.09 ± 0.14 | | | 25.04 ± 0.13 | | |
| SD | | | | | | | | | |

One skilled in the art would expect that recovery of target cfDNA should decrease greatly with increasing number of ATPS, as there are more potential sources of cfDNA loss, such as loss due to manual handling and pipetting errors. However, referring now to FIG. 2A and Table 5, all conditions (Groups 1-3) have average Ct values for 145 bp dsDNA that are within 0.2 Ct of each other, indicating no major loss of target cfDNA despite increased number of ATPS used to process the sample. The results show that splitting a large volume sample into multiple smaller ATPS in parallel surprisingly retains cfDNA yield and does not affect performance when compared to a one single large ATPS.

### Example 6: Urine Extraction by Splitting Sample Matrix into 4× First ATPS

In this example, urine samples were split into 4 separate first ATPS, and further went through 2 separate second ATPS or a single second ATPS. The DNA recovery thereof was compared.

### Urine Lysis

In this example, urine samples were pre-treated by the same procedures discussed in Example 5. To digest unwanted protein and cells present in pre-treated urine samples, 2400 μL of Proteinase K (28.57 mg/mL) and 8 mL of suitable lysis buffer were added to 80 mL of sample. 100 fg of 145 bp dsDNA and 100 ng of 1 kb+DNA ladder was spiked into the above sample. The samples were then vortexed thoroughly till homogenous then left in a pre-heated 37° C. water bath to incubate for 15 minutes.

### Two Phase System

The extraction procedure is similar to the procedures discussed in Example 5. In the first ATPS, DNA partitions to the bottom phase and proteins partition to the top phase. The bottom phase, which amounts to around 5 mL, was carefully extracted and transferred to the second ATPS. In the second ATPS, DNA partitions strongly to the top phase, which amounts to around 500 uL, effectively concentrating 20 mL of sample matrix into 500 μL of polymer-rich phase containing target cfDNA. In this experiment we demonstrated that usage of multiple smaller ATPS in parallel does not affect performance when compared to a one single large ATPS. Urine samples from 2 donors were split into 2 separate groups (Groups 4 and 5). For Group 4, 90.4 mL of urine sample were split evenly into 4 separate first ATPS (4× first ATPS). The top phases from 2 out of 4 first ATPS were combined and added to 1 second ATPS, resulting in 2 separate second ATPS in total (2× second ATPS). For Group 5, 90.4 mL of urine sample were similarly split evenly into 4 separate first ATPS, however, all of the top phases from these first ATPS were combined and added to 1 single larger second ATPS (1× second ATPS). To ensure an equal comparison, second ATPS were scaled accordingly depending on conditions to ensure compositions of salt and polymer were identical between conditions. The extraction conditions of the first and second ATPS used in this experiment are summarized in Table 6. The concentration of polymer and salts and/or the volume the first and second ATPS compositions were adjusted accordingly depending on the volume of the lysates used (22.6 mL in this example) accordingly to Table 6.

TABLE 6
Overall summary of workflow between different ATPS conditions.

| | | Scale | | Scale |
|---|---|---|---|---|
| Group | First ATPS | factor | Second ATPS | factor |
| 4 | 4x first ATPS | 1x | 2x second ATPS | 1x |
| | (each containing | | (each containing 10 mL first | |
| | 22.6 mL lysate) | | ATPS bottom phase) | |
| 5 | | | 1x second ATPS | 2x |
| | | | (containing 20 mL first ATPS | |
| | | | bottom phase) | |

TABLE 7
Concentrations of polymer and salt in both
first and second ATPS respectively.

| First ATPS | Second ATPS |
|---|---|
| 20-40% PAG, 0.1-20% phosphates | 3-20% PAG, 0.1-20% phosphates |

All concentrations are in w/v ratio.

### Purification of DNA

The purification procedures used in this example are the same as those discussed in Example 5 except 4 mL of binding buffer (3-7M guanidinium) was used. For the sake of brevity and simplicity of the present disclosure, the discussion of the purification steps is not reproduced here.

### Detection of DNA

The steps to perform the detection of DNA for each sample are the same or similar to those as discussed with respect to Example 4a above. For the sake of brevity and simplicity of the present disclosure, the discussion of the detection steps is not reproduced here. The results were presented as average CT values.

### Results

FIG. 2B shows the Ct values obtained from qPCR of 145 bp dsDNA recovered under different extraction conditions (Groups 4-5) according to Table 6. The results are also summarized in Table 8 below.

All conditions have Ct values for 145 bp dsDNA that are within 0.1 Ct of each other, indicating no major loss of target cfDNA with increased number of ATPS.

TABLE 8
Average Ct values from qPCR of 145 bp dsDNA recovered.

| | 4x ATPS#1 + 2x ATPS#2Group | | 4x ATPS#1 + 1x ATPS#2Group | |
|---|---|---|---|---|
| | 4 | | 5 | |
| | Donor 1 | Donor 2 | Donor 1 | Donor 2 |
| CT value (145 bp) | 25.15 | 25.15 | 25.08 | 25.20 |

| | | |
|---|---|---|
| Average ± SD | 25.15 ± 0 | 25.16 ± 0.07 |

Referring now to FIG. 2B and Table 8, all conditions (Groups 4-5) have Ct values for 145 bp dsDNA that are within 0.1 Ct of each other, indicating no major loss of target cfDNA with increased number of second ATPS. The results show that processing the samples extracted from first ATPS in multiple smaller second ATPS in parallel surprisingly retains cfDNA yield and does not affect performance when compared to one single second ATPS.

### Example 7: Urine Extraction Using ATPS with Extreme Volume Ratios

This experiment tests the robustness of DNA recovery over a range of different volume ratios in the 1^{st} ATPS and the 2^{nd} ATPS. In this example, 0.25×PBS and urine samples were tested with different ATPS conditions which resulted in different phase volume ratios.

### Urine Lysis

In this example, one set of data was produced using 0.25×PBS as sample matrix, another set used urine samples from three individual donors. Similar to Example 5 and 6, the samples were pre-treated with 200 μL of 0.1 M EDTA per 10 mL urine sample, vortexed thoroughly and centrifuged at 3000 rcf for 10 minutes. The supernatant was transferred to a new tube while the pellet was discarded. To digest unwanted protein and cells present in pre-treated urine samples, 2400 μL of Proteinase K (28.57 mg/mL) and 8 mL of suitable lysis buffer were added to 80 mL of sample. 100 fg of 145 bp dsDNA and 100 ng of 1 kb+DNA ladder was spiked into the above sample. The samples were then vortexed thoroughly till homogenous then left in a pre-heated 37° C. water bath to incubate for 15 minutes.

### Two Phase System

The extraction procedure involves two sequential aqueous two-phase systems (ATPS) to isolate, purify and concentrate DNA from a urine sample. 22.6 mL of lysate were transferred into the first ATPS, where DNA partitions to the bottom phase and proteins partition to the top phase. The bottom phase was carefully extracted and transferred to the second ATPS. In the second ATPS, DNA partitions strongly to the top phase effectively concentrating more than 20 mL of sample matrix into a much smaller polymer-rich phase containing target cfDNA.

This experiment has been split into two parts: variation in top:bottom volume ratio in 1^{st} ATPS while keeping the 2^{nd} ATPS volume ratio constant while another part focuses on keeping the 1^{st} ATPS constant while varying the volume ratio of the 2^{nd} ATPS. To test whether DNA recovery would be affected by variations in the 1^{st} ATPS phase volume ratio, various formulas giving a range of top:bottom phase volume ratio from 0.9:1 to 13:1 were tested, while keeping 2^{nd} ATPS volume ratio constant at 1:24. Details of formulas are summarized in Table 9. The concentration of polymer and salts and the volume the first and second ATPS compositions were adjusted accordingly such that the 1^{st} ATPS formed the respective top:bottom volume ratios of 13:1, 6:1 and 0.9:1 after mixing with 22.6 mL lysate, and the 2^{nd} ATPS formed the constant top:bottom volume ratio of 1:24 in all three conditions after mixing with the various volumes of the bottom phase from the 1^{st} ATPS (condition 1=2.6 mL, condition 2=5 mL, and condition 3=20 mL).

TABLE 9
Formulas tested to validate 1^{st} ATPS volume ratio variability

| | 1^{st} ATPS | 2^{nd} ATPS |
|---|---|---|
| | top:bottom | top:bottom |
| Condition* | volume ratio | volume ratio |
| 1 | 13:1 | 1:24 |
| 2 | 6:1 | 1:24 |
| 3 | 0.9:1 | 1:24 |

*All 1^{st} ATPS compositions contain PAG, phosphates, 1 mM EDTA and 0.6% Triton X-114, and all 2^{nd} ATPS compositions contain PAG, phosphates, and 0.7 mM EDTA.

To test whether DNA recovery would be affected by varying 2^{nd} ATPS top:bottom phase volume ratio, 1^{st} ATPS formula is kept constant and 2^{nd} ATPS formulas giving 1:1 and 1:24 are tested (Table 10). The concentration of polymer and salts and the volume the first and second ATPS compositions were adjusted accordingly such that the 1st ATPS formed a bottom phase of about 3.5-6 mL and a top:bottom volume ratio of 6:1 after mixing with 22.6 mL lysate, and the 2nd ATPS formed the respective top:bottom volume ratios of 1:24 and 1:1 after mixing with the bottom phase from the 1st ATPS, which was about 5 mL in this example.

TABLE 10
Formulas tested to validate 1^{st} ATPS volume ratio variability

| | 1^{st} ATPS | 2^{nd} ATPS |
|---|---|---|
| | Top:Bottom | Top:Bottom |
| Condition* | volume ratio | volume ratio |
| 4 | 6:1 | 1:24 |
| 5 | | 1:1 |

*All 1^{st} ATPS compositions contain PAG, phosphates, 1 mM EDTA and 0.6% Triton X-114, and all 2^{nd} ATPS compositions contain PAG, phosphates, and 0.7 mM EDTA.

### Purification of DNA

Similar to the procedures discussed in Examples 5 and 6, the purification of DNA from the extracted phase was done by spin column extraction. The top phase from the second ATPS was transferred to a tube containing binding buffer (3-7M guanidinium) of which amount is scaled to top phase volume accordingly at 1:4 phase to binding buffer ratio. The extracted top phase and binding buffer were mixed thoroughly, 800 μL of the solution was transferred to a spin column (EconoSpin) and was centrifuged for 30 seconds at 12,000 rcf. The flow-through was discarded. The process was repeated until all samples were passed through the spin column. 500 μL of washing buffer (80% ethanol v/v, 0.1 M NaCl, 0.01 M Tris-HCl) was added to the spin column, and the spin column was centrifuged for 30 seconds at 12,000 rcf. The spin column was then centrifuged for 2 minutes at 16,000 rcf to dry. 40 μL of elution buffer (0.01 M Tris-HCl, 0.001 M EDTA) was added to the spin column membrane. The spin column was incubated at room temperature for 3 minutes. The elution was collected to a collection tube by centrifuge for 1 minute at 12,000 rcf for detection.

### Detection of DNA

DNA detection was performed by the same method discussed in preceding examples.

### Results

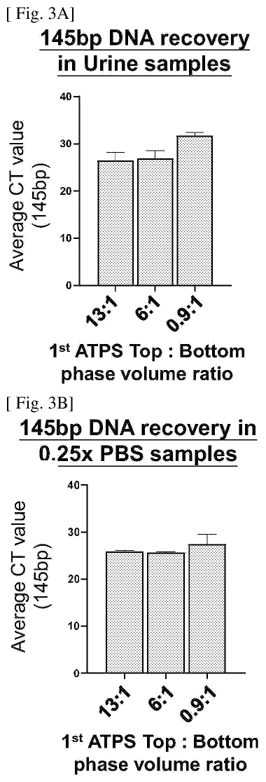

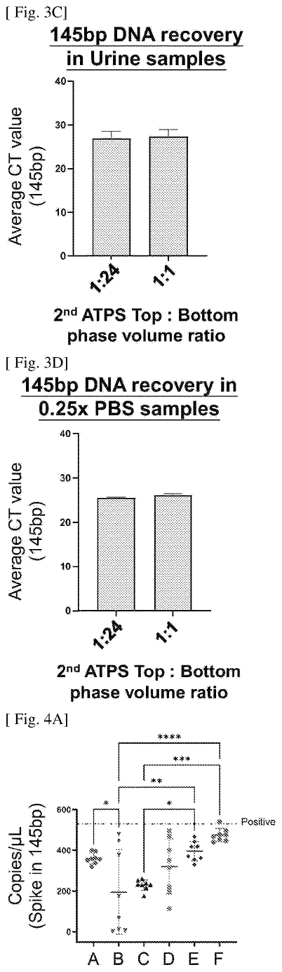

Now referring to FIGS. 3A-3D, the resulting DNA recovery using different volume ratios, according to the conditions in Table 9 and 10 (the respective first ATPS volume ratios as in Conditions 1-3 and the respective second ATPS volume ratios as in Conditions 4-5), are shown. FIG. 3A shows the recovery of 145 bp dsDNA spike-in using 1^{st} ATPS with varying top:bottom phase volume ratio with urine samples from three individual donors. The results are also summarized in Table 11. FIG. 3B shows the recovery of 145 bp dsDNA spike-in using 1^{st} ATPS with varying top:bottom phase volume ratio with 0.25×PBS as sample matrix. The results are also summarized in Table 12.

TABLE 11
Average CT values of 145 bp DNA in urine samples.

| | | 1^{st} ATPS Top:Bottom volume ratio | | |
|---|---|---|---|---|
| | | 13:1 | 6:1 | 0.9:1 |
| CT value | Donor 1 | 25.282 | 26.050 | 30.942 |
| (145 bp) | Donor 2 | 28.429 | 25.978 | 32.007 |
| | Donor 3 | 25.908 | 28.748 | 32.285 |

| | | | |
|---|---|---|---|
| Avg. ± SD | 26.54 ± 1.7 | 26.93 ± 1.6 | 31.74 ± 0.7 |

TABLE 12
Average CT values of 145 bp DNA in 0.25x PBS.

1^{st} ATPS Top:Bottom volume ratio

| | 13:1 | 6:1 | 0.9:1 |
|---|---|---|---|
| CT value | 25.967 | 25.428 | 25.756 |
| (145 bp) | 25.693 | 25.693 | 28.916 |
| Avg. ± SD | 25.83 ± 0.2 | 25.56 ± 0.2 | 27.34 ± 2.2 |

FIG. 3C shows the recovery of 145 bp dsDNA spike-in using 2^{nd} ATPS with varying top:bottom phase volume ratio with urine samples from three individual donors. The results are also summarized in Table 13. FIG. 3D shows the recovery of 145 bp dsDNA spike-in using 2^{nd} ATPS with varying top:bottom phase volume ratio with 0.25×PBS as sample matrix. The results are also summarized in Table 14.

TABLE 13
Average CT values of 145 bp DNA in urine samples.

2^{nd} ATPS Top:Bottom volume ratio

| | | 1:24 | 1:1 |
|---|---|---|---|
| CT value | Donor 1 | 26.050 | 25.872 |
| (145 bp) | Donor 2 | 25.978 | 29.096 |
| | Donor 3 | 28.748 | 28.748 |

| | | |
|---|---|---|
| Avg. ± SD | 26.93 ± 1.6 | 27.91 ± 1.8 |

TABLE 14
Average CT values of 145 bp DNA in 0.25x PBS.

2^{nd} ATPS Top:Bottom volume ratio

| | 1:24 | 1:1 |
|---|---|---|
| CT value | 25.428 | 25.693 |
| (145 bp) | 26.393 | 25.98 |
| Avg. ± SD | 25.91 ± 0.7 | 25.84 ± 0.2 |

The results demonstrate that the ATPS system works across different volume ratios, and works particularly well in certain volume ratios. This experiment demonstrated that DNA could still be recovered by changing the top:bottom phase volume ratio in 1^{st} ATPS (FIGS. 3A and 3B) and 2^{nd} ATPS (FIGS. 3C and 3D). The recovery of DNA in all the ATPS systems tested with spin columns performed better than in the systems having no ATPS (see urine Sample #2 in Example 4a).

The above examples have demonstrated the robustness and stability of ATPS in the various cases where large volume or bulk fluid samples are handled. This highlights the advantages of the methods, kits, and embodiments described in the current disclosure, which can be used or adapted by any persons skilled in the art in different settings to achieve comparable DNA recovery from large volume or bulk fluid samples, such as urine, while minimizing errors and sample loss typically associated with sample handling and processing.

### Example 8: Comparison of Total DNA Recovery Using the Presently Disclosed Method to Commercially Available Extraction Kits

### Example 8a

DNA extraction from urine using an exemplary method and kit (referred as ‘present extraction method’ or ‘Phase’ herein) as described in Example 4a was compared to that of the Zymo Quick-DNA Urine kit (‘Zymo’), NextPrep-Mag Urine cfDNA Isolation Kit (‘NextPrep kit’), Norgen Urine DNA Isolation kit-spin column (‘Norgen’), and Wizard Plus miniprep DNA purification system (‘Wizard’), which are all commercially available. For each commercially available kit, the maximum input volume of urine sample as specified by the manufacturer was used, and the extraction was performed by following the manufacturer's instruction. The comparison was performed using cell-free urine for the commercially available kits (Conditions A-D in Table 15) as well as the present extraction method (Condition E in Table 15). Additionally, as a comparison, we performed one batch of 40 mL extraction using crude urine (unspun urine with cells) with the present extraction method (Condition F in Table 15) to assess if the present extraction method can perform equally well with the cells. Urine samples were provided by 4 males and 4 females (n=8 for each kit). The input and elution volume of each kit were normalized to a 100:1 ratio, and the extraction time is compared in Table 15.

TABLE 15
Comparisons of yield and efficiency with normalized input:elution volume ratio

| | | | | | Average 145 bp |
|---|---|---|---|---|---|
| | Extraction | Input | Elution | Extraction | DNA recovery |
| Cond. | system | volume | volume | time | (copies/uL) ± SD |
| A | NextPrep kit | 20 mL | 200 μL | 120 min | 360.9 ± 27.2 |
| B | Zymo | 40 mL | 400 μL | 160 min | 196.1 ± 207.4 |
| C | Norgen | 30 mL | 300 μL | 175 min | 230.1 ± 25.5 |
| D | Wizard | 10 mL | 100 μL | 178 min | 321.6 ± 135.7 |
| E | 40 mL Phase | 40 mL | 400 μL | 143 min | 396.8 ± 48.0 |
| F | 40 mL Phase | 40 mL | 400 μL | 133 min | 476.1 ± 32.2 |
| | [Crude urine] | | | | |

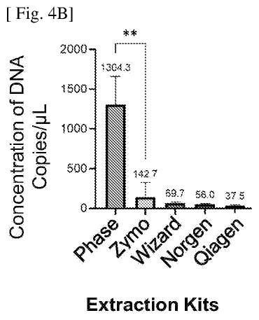

Now referring to FIG. 4A, which shows the recovery of 145 bp DNA spike-in (copies/uL) using the conditions A-F according to Table 15. Detection of 145 bp spike-in was performed by Droplet Digital PCR ddPCR. As shown in FIG. 4A, the 145 bp spike-in DNA recovery efficiency using the present extraction method (conditions E) was comparable if not greater than that of the NextPrep (condition A) and Wizard (condition D) extraction kits and significantly higher than that of the Zymo (condition B) and Norgen (condition C) extraction kits. Compared to the NextPrep (condition A), Norgen (condition C) and Wizard (condition D) extraction kits, the present extraction method can process a larger input volume but with a comparable if not shorter extraction time. Compared to Zymo (Condition B) with the same input volume, the present extraction method has a shorter extraction time with a significantly higher yield as well as a much more consistent sample-to-sample performance. Whilst using Urine with cells (condition F), the average DNA recovery by the present extraction method was significantly better than all of the commercially available kits with satisfactory precision (476.1±32.2 copies/uL) even with the presence of cells. This shows that the present extraction method performed well in recovering target DNA with crude urine as well as processed spun down urine. In summary, the overall target DNA extraction performance (in terms of yield, input volume and extraction time) using the method of the present disclosure is surprisingly better compared to the industry standard, commercially available extraction kits.

### Example 8b

Further comparison of DNA recovery from urine between the present extraction method and the commercially available extraction kits (Zymo Quick-DNA Urine kit (‘Zymo’), Qiagen QIAamp Circulating Nucleic Acid Kit (‘Qiagen’ or ‘QCNA’), Norgen Urine DNA Isolation kit-spin column (‘Norgen’), and Wizard Plus miniprep DNA purification system (‘Wizard’)) was performed by using the present extraction method (also referred as “Phase” herein) with a maximum urine sample input volume of 160 mL, while the commercially available extraction kits utilized the maximum sample input volume and optimal output volume recommended by the manufacturer, and the extraction was performed by following the manufacturer's instruction. Urine samples were provided by 4 males and 4 females (n=8 for each kit). The conditions are summarized in Table 16.

TABLE 16
Comparisons of yield and efficiency using
the recommended input and output volume

| | Input | Output | Average 145 bp DNA |
|---|---|---|---|
| Extraction Kit | Volume | Volume | recovery (copies/uL) |
| Present extraction | 160 mL | 20 μL | 1304.3 |
| method (Phase) | | | |
| Zymo | 40 mL | 10 μL | 142.7 |
| QCNA (Qiagen) | 4 mL | 20 μL | 69.7 |
| Wizard | 10 mL | 50 μL | 56.0 |
| Norgen | 30 mL | 50 μL | 37.5 |

Now referring to FIG. 4B, which shows the average concentration of recovered DNA (copies/uL) using the kits and conditions according to Table 16. Detection of 140 bp spike-in was performed by Droplet Digital PCR ddPCR. The results demonstrated that the present extraction method was able to significantly out-perform all the commercially available kits due to the ability to process high input volume and concentrate it to a low output volume.

In summary, the experiments in Examples 8a and 8b demonstrate that the total DNA recovery of the present extraction method is significantly greater than all the other commercially available kits, due to the both the increased allowable sample input volume of the present extraction method and the low output/elution volume as well as the greater recovery efficiency of target DNA.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### NUMBERED EMBODIMENTS 1

### Embodiment 1

A method for concentrating and purifying one or more target analytes from a bulk fluid sample, comprising the steps of: (a) preparing a first aqueous two-phase system (ATPS) composition, wherein the first ATPS composition comprises polymers, salts, surfactants, or combinations thereof dissolved in an aqueous solution to form a first phase solution and a second phase solution; (b) adding a sample solution prepared from the bulk fluid sample containing the target analyte (s) to the first ATPS composition, such that the target analyte (s) partition to the first phase solution; (c) collecting the first phase solution and mixing the first phase solution with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution; (d) collecting the third phase solution and mixing the third phase solution with a binding buffer to form a mixed solution, wherein the binding buffer comprises at least one chaotropic agent; I loading the mixed solution onto an extraction column configured to selectively extract and purify the target analyte (s); (f) eluting and collecting the target analyte (s) from the extraction column, resulting in a final solution containing the concentrated and purified target analyte (s).

### Embodiment 2

The method of embodiment 1, wherein the sample solution is prepared by dividing the bulk fluid sample containing the target analyte (s) into at least two aliquots of the sample solution, and the first ATPS composition is divided into at least two aliquots, wherein step (b) further includes the following steps: (i) adding each aliquot of said sample solution prepared from the bulk fluid sample containing the target analyte (s) to each aliquot of the first ATPS composition, such that the target analyte (s) partition to the first phase solution; (ii) collecting and combining the first phase solutions of the at least two aliquots of the first ATPS composition to form the first phase solution for step (c).

### Embodiment 3

The method of any of the preceding embodiments, wherein the extraction column is a spin column, and wherein step (e) further comprises the following steps: (i) loading a portion of the mixed solution onto the extraction column; (ii) centrifuging the extraction column and discarding the flow-through or supernatant; and (iii) repeating steps (i) and (ii) above until all of the mixed solution has been passed through the extraction column.

### Embodiment 4

The method of any of the preceding embodiments, further comprising the step of: (g) subjecting said final solution to a diagnostic assay for detection and quantification of said target analyte (s).

### Embodiment 5

A method for concentrating and purifying one or more target analytes from a bulk fluid sample, comprising the steps of: (a) dividing the bulk fluid sample containing the target analyte (s) into at least two aliquots of a sample solution; (b) preparing at least two aliquots of a first aqueous two-phase system (ATPS) composition, wherein the first ATPS composition comprises polymers, salts, surfactants, or combinations thereof dissolved in an aqueous solution to form a first phase solution and a second phase solution; (c) adding each aliquot of said sample solution containing the target analyte (s) to each aliquot of the first ATPS composition, such that the target analyte (s) partition to the first phase solution; (d) collecting the first phase solutions of the at least two aliquots of the first ATPS composition, and mixing the first phase solutions with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution; (e) collecting the third phase solution and mixing the third phase solution with a binding buffer to form a mixed solution, wherein the binding buffer comprises at least one chaotropic agent; (f) loading the mixed solution onto an extraction column configured to selectively extract and purify the target analyte (s); (g) eluting and collecting the target analyte (s) from the extraction column.

### Embodiment 6

The method of any one of the preceding embodiments, wherein the bulk fluid sample is selected from the group consisting of blood, plasma, serum, cerebrospinal fluid, urine, saliva, fecal matter, tear, sputum, nasopharyngeal mucus, vaginal discharge and penile discharge.

### Embodiment 7

The method of any one of the preceding embodiments, wherein the bulk fluid sample is urine.

### Embodiment 8

The method of any one of the preceding embodiments, wherein the bulk fluid sample has a volume of 40 mL or more.

### Embodiment 9

The method of any one of the preceding embodiments, wherein each aliquot of said sample solution has a volume of up to 40 ml.

### Embodiment 10

The method of any one of the preceding embodiments, wherein the target analytes are selected from the group consisting of nucleic acids, a protein, an antigen, a biomolecule, a sugar moiety, a lipid, a sterol, and combinations thereof.

### Embodiment 11

The method of any one of the preceding embodiments, wherein the target analytes are DNA.

### Embodiment 12

The method of any one of the preceding embodiments, wherein the target analytes are cell-free DNA or circulating tumor DNA.

### Embodiment 13

The method of any one of the preceding embodiments, wherein said polymers are dissolve in the aqueous solution at a concentration of 4%-84% (w/w).

### Embodiment 14

The method of any one of the preceding embodiments, wherein said polymers are selected from the group consisting of polyalkylene glycols, such as hydrophobically modified polyalkylene glycols, poly (oxyalkylene) polymers, poly (oxyalkylene) copolymers, such as hydrophobically modified poly (oxyalkylene) copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. The method of any one of the preceding embodiments, wherein the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymer thereof. The method of any one of the preceding embodiments, wherein the polymer is selected from the group consisting of dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide and copolymers thereof. The method of any one of the preceding embodiments, wherein the polymer is selected from the group consisting of dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether and poly N-isopropylacrylamide. The method of any one of the preceding embodiments, wherein the polymer is selected from the group consisting of polyacrylamide, polyacrylic acid and copolymers thereof. The method of any one of the preceding embodiments, wherein the polymer is selected from the group consisting of dextran, carboxymethyl dextran, dextran sulfate, hydroxypropyl dextran and starch. The method of any one of the preceding embodiments, wherein the polymer has an average molecular weight in the range of 200-1,000 Da, 200-35,000 Da, 425-2,000 Da, 400-35,000 Da, 980-12,000 Da, or 3, 400-5,000,000 Da. The method of any one of the preceding embodiments, wherein the polymer comprises ethylene oxide and propylene oxide units, and the polymer has an EO:PO ratio of 90:10 to 10:90.

### Embodiment 15

### Embodiment 16

The method of any one of the preceding embodiments, wherein said salts are dissolved in the aqueous solution at a concentration of 1%-55% (w/w).

### Embodiment 17

The method of any one of the preceding embodiments, wherein said salts are dissolved in the aqueous solution at a concentration of 8%-55% (w/w).

### Embodiment 18

The method of any one of the preceding embodiments, wherein said salts are selected from the group consisting of kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate, NaCl, Na₃PO₄, K₃PO₄, Na₂SO₄, potassium citrate, (NH₄)₂SO₄, sodium citrate, sodium acetate, ammonium acetate, a magnesium salt, a lithium salt, a sodium salt, a potassium salt, a cesium salt, a zinc salt, an aluminum salt, a bromide salt, an iodide salt, a fluoride salt, a carbonate salt, a sulfate salt, a citrate salt, a carboxylate salt, a borate salt, a phosphate salt, potassium phosphate and ammonium sulfate.

### Embodiment 19

The method of any one of the preceding embodiments, wherein said surfactants are dissolved in the aqueous solution at a concentration of 0.05%-10% (w/w).

### Embodiment 20

The method of any one of the preceding embodiments, wherein said surfactants are dissolved in the aqueous solution at a concentration of 0.05%-9.8% (w/w).

### Embodiment 21

The method of any one of the preceding embodiments, wherein said surfactants are selected from the group consisting of Triton-X, Triton-114, Igepal CA-630 and Nonidet P-40, anionic surfactants, such as carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, alkylphenols, ethoxylated and sulphated, nonionic surfactants, such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants, such as quaternary ammonium salts, amines with amide linkages, polyoxyethylene alkyl & alicyclic amines, n, ′, n′, n′tetrakis substituted ethylenediamines, 2-alkyl 1-hydroxethyl 2-imidazolines, and amphoteric surfactants, such as n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt.

### Embodiment 22

An ATPS composition selected from the group consisting of A1, A2, A3, A4, AA1, AA2, AA3, and AA4.

### Embodiment 23

A kit comprising a first ATPS composition selected from the group consisting of A1, A2, A3, and A4; a second ATPS composition selected from the group consisting of AA1, AA2, AA3, and AA4; and a binding buffer selected from the group consisting of B1, B2, and B3.

### Embodiment 24

The kit of embodiment 23, further comprises an extraction column.

### NUMBERED EMBODIMENTS 2

### Embodiment 1

A method for concentrating and purifying one or more target analyte (s) from a bulk fluid sample, comprising the steps of: (a) dividing the bulk fluid sample containing the target analyte (s) into at least two aliquots of a sample solution; (b) preparing at least two first aqueous two-phase system (ATPS) compositions, wherein each first ATPS composition comprises a polymer, a salt, a surfactant, or any combination thereof dissolved in an aqueous solution to form a first phase solution and a second phase solution; (c) adding each aliquot of said sample solution prepared from the bulk fluid sample containing the target analyte (s) to each first ATPS composition, such that the target analyte (s) partition to each first phase solution; (d) further processing each first phase solution to form a final phase solution; (e) mixing the final phase solution with at least one purifying composition to form a mixed solution; (f) contacting the mixed solution with a downstream purification system configured to selectively isolate the target analyte (s); and (g) collecting the target analyte (s) from the downstream purification system, resulting in a final solution containing the concentrated and purified target analyte (s).

### Embodiment 2

The method of any one of the preceding embodiments, wherein the further processing of step (d) comprises collecting and combining each first phase solution to form the final phase solution.

### Embodiment 3

The method of any one of the preceding embodiments, wherein the further processing of step (d) comprises the steps of (i) collecting each first phase solution; (ii) mixing each first phase solution with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution; (iii) collecting and combining the third phase solutions to form the final phase solution.

### Embodiment 4

The method of any one of the preceding embodiments, wherein further processing of step (d) comprises the steps of (i) collecting and combining each first phase solution to form a combined first phase solution; (ii) mixing each combined first phase solution with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution; (iii) collecting the third phase solution to form the final phase solution.

### Embodiment 5

The method of any one of the preceding embodiments, wherein the purifying composition is a binding buffer comprising at least one chaotropic agent; the downstream purification system comprises a solid phase medium; and step (f) further comprises the following steps: (i) contacting a portion of the mixed solution with the solid phase medium such that the target analyte (s) binds to the solid phase medium to form a solid phase extraction complex; (ii) perturbing the solid phase extraction complex and discarding the flow-through or supernatant; and (iii) optionally repeating steps (i) and (ii).

### Embodiment 6

The method of any one of the preceding embodiments, wherein the solid phase medium is a solid phase extraction column.

### Embodiment 7

The method of any one of the preceding embodiments, wherein the solid phase extraction column is a spin column.

### Embodiment 8

The method of any one of the preceding embodiments wherein the solid phase medium is a plurality of beads.

### Embodiment 9

The method of any one of the preceding embodiments, wherein the plurality of beads is magnetic beads, silica-based beads, carboxyl beads, hydroxyl beads, amine-coated beads, or any combination thereof.

### Embodiment 10

The method of any one of the preceding embodiments, further comprising the step of: (h) subjecting said final solution to a diagnostic assay for detection, quantification, characterization, or combinations thereof, of said target analyte (s).

### Embodiment 11

A method for concentrating and purifying one or more target analytes from a bulk fluid sample, comprising the steps of: (a) dividing the bulk fluid sample containing the target analyte (s) into at least two aliquots of a sample solution; (b) preparing at least two aliquots of a first aqueous two-phase system (ATPS) composition, wherein the first ATPS composition comprises polymers, salts, surfactants, or combinations thereof dissolved in an aqueous solution to form a first phase solution and a second phase solution; (c) adding each aliquot of said sample solution containing the target analyte (s) to each aliquot of the first ATPS composition, such that the target analyte (s) partition to the first phase solution; (d) collecting the first phase solutions of the at least two aliquots of the first ATPS composition, and mixing the first phase solutions with a second ATPS composition, wherein the second ATPS composition comprises polymers, salts, surfactants, or combinations thereof dissolved in an aqueous solution to form a third phase solution and a fourth phase solution, such that the target analyte (s) partition to and concentrate in the third phase solution; (e) collecting the third phase solution and mixing the third phase solution with a binding buffer to form a mixed solution, wherein the binding buffer comprises at least one chaotropic agent; (f) loading the mixed solution onto an extraction column configured to selectively extract and purify the target analyte (s); (g) eluting and collecting the target analyte (s) from the extraction column.

### Embodiment 12

The method of any one of the preceding embodiments, wherein the bulk fluid sample is selected from the group consisting of blood, plasma, serum, cerebrospinal fluid, urine, saliva, fecal matter, tear, sputum, nasopharyngeal mucus, vaginal discharge and penile discharge.

### Embodiment 13

The method of any one of the preceding embodiments, wherein the bulk fluid sample is urine.

### Embodiment 14

The method of any one of the preceding embodiments, wherein the bulk fluid sample has a volume of at least 10 mL.

### Embodiment 15

The method of any one of the preceding embodiments, wherein the bulk fluid sample has a volume of 40 mL or more.

### Embodiment 16

The method of any one of the preceding embodiments, wherein each aliquot of said sample solution has a volume of up to 40 ml.

### Embodiment 17

The method of any one of the preceding embodiments, wherein each aliquot of said sample solution has a volume of 10 to 40 mL.

### Embodiment 18

The method of any one of the preceding embodiments, wherein the target analyte (s) is selected from the group consisting of a nucleic acid, a protein, an antigen, a biomolecule, a sugar moiety, a lipid, a sterol, and any combination thereof.

### Embodiment 19

The method of any one of the preceding embodiments, wherein the target analyte (s) is DNA.

### Embodiment 20

The method of any one of the preceding embodiments, wherein the target analyte (s) is gDNA, cDNA, plasmid DNA, mitochondrial DNA, cell-free DNA (cfDNA), circulating tumor DNA (ctDNA), circulating fetal DNA, cell-free microbial DNA, micro RNA (miRNA), messenger RNA (mRNA), transfer RNA (tRNA), ribosomal RNA (rRNA), circular RNA, long non-coding RNA (lncRNA) or combinations thereof.

### Embodiment 21

The method of any one of the preceding embodiments, wherein the target analyte (s) is cell-free DNA (cfDNA) or circulating tumor DNA (ctDNA).

### Embodiment 22

The method of any one of the preceding embodiments, wherein the polymer is dissolved in an aqueous solution at a concentration of 0.2-80% (w/v).

### Embodiment 23

The method of any one of the preceding embodiments, wherein the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymer thereof.

### Embodiment 24

The method of any one of the preceding embodiments, wherein the polymer is selected from the group consisting of dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide and copolymers thereof.

### Embodiment 25

The method of any one of the preceding embodiments, wherein the polymer is selected from the group consisting of dipropylene glycol, tripropylene glycol, polyethylene glycol, polypropylene glycol, poly (ethylene glycol-propylene glycol), poly (ethylene glycol-ran-propylene glycol), polyvinyl pyrrolidone, polyvinyl alcohol, polyvinyl caprolactam, polyvinyl methylether and poly N-isopropylacrylamide.

### Embodiment 26

The method of any one of the preceding embodiments, wherein the polymer is selected from the group consisting of polyacrylamide, polyacrylic acid and copolymers thereof.

### Embodiment 27

The method of any one of the preceding embodiments, wherein the polymer is selected from the group consisting of dextran, carboxymethyl dextran, dextran sulfate, hydroxypropyl dextran and starch.

### Embodiment 28

The method of any one of the preceding embodiments, wherein the polymer has an average molecular weight in the range of 200-1,000 Da, 200-35,000 Da, 425-2,000 Da, 400-35,000 Da, 980-12,000 Da, or 3, 400-5,000,000 Da.

### Embodiment 29

The method of any one of the preceding embodiments, wherein the polymer comprises ethylene oxide and propylene oxide units, and the polymer has an EO:PO ratio of 90:10 to 10:90.

### Embodiment 30

The method of any one of the preceding embodiments, wherein the salt is dissolved in an aqueous solution at a concentration of 0.1% to 80% (w/v).

### Embodiment 31

The method of any one of the preceding embodiments, wherein the salt comprises a cation selected from the group consisting of sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium and tetrabutyl ammonium.

### Embodiment 32

The method of any one of the preceding embodiments, wherein the salt comprises an anion selected from the group consisting of phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, and tris.

### Embodiment 33

The method of any one of the preceding embodiments, wherein the salt is selected from the group consisting of aluminum chloride, aluminum phosphate, aluminum carbonate, magnesium chloride, magnesium phosphate, and magnesium carbonate.

### Embodiment 34

The method of any one of the preceding embodiments, wherein salt is selected from the group consisting of NaCl, KCl, NH₄Cl, Na₃PO₄, K₃PO₄, Na₂SO₄, K₂HPO₄, KH₂PO₄, Na₂HPO₄, NaH₂PO₄, (NH₄)₃PO₄, (NH₄)₂HPO₄, NH₄H₂PO₄, potassium citrate, (NH₄)₂SO₄, sodium citrate, sodium acetate, magnesium acetate, sodium oxalate, sodium borate, and ammonium acetate.

### Embodiment 35

The method of any one of the preceding embodiments, wherein salt is selected from the group consisting of (NH₄)₃PO₄, sodium formate, ammonium formate, K₂CO₃, KHCO₃, Na₂CO₃, NaHCO₃, MgSO₄, MgCO₃, CaCO₃, CsOH, Cs₂CO₃, Ba(OH)₂, and BaCO₃.

### Embodiment 36

The method of any one of the preceding embodiments, wherein salt is selected from the group consisting of NH₄Cl, NH₄OH, tetramethyl ammonium chloride, tetrabutyl ammonium chloride, tetramethyl ammonium hydroxide, and tetrabutyl ammonium hydroxide.

### Embodiment 37

The method of any one of the preceding embodiments, wherein the surfactant is dissolved in an aqueous solution at a concentration of 0.05%-10% (w/v).

### Embodiment 38

The method of any one of the preceding embodiments, wherein the surfactant is selected from the group consisting of anionic surfactant, nonionic surfactant, cationic surfactant, and amphoteric surfactant; and wherein the anionic surfactant is carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, or sodium N-lauroyl sarcosinate (NLS); the nonionic surfactant is ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, or polyoxyethylene fatty acid amides; the cationic surfactant is quaternary ammonium salts, amines with amide linkages, polyoxyethylene alkyl amines, polyoxyethylene alicyclic amines, n, n, n′, n′tetrakis substituted ethylenediamines, or 2-alkyl 1-hydroxethyl 2-imidazolines; and the amphoteric surfactant is n-coco 3-aminopropionic acid or sodium salt thereof, n-tallow 3-iminodipropionate or disodium salt thereof, n-carboxymethyl n dimethyl n-9 octadecenyl ammonium hydroxide, or n-cocoamidethyl n hydroxyethylglycine or sodium salt thereof.

### Embodiment 39

The method of any one of the preceding embodiments, wherein the surfactant is Triton X-100, Triton X-114, Triton X-45, Tween 20, Igepal CA630, Brij 58, Brij O10, Brij L23, Pluronic L-61, Pluronic F-127, sodium dodecyl sulfate, sodium cholate, sodium deoxycholate, sodium N-lauroyl sarcosinate (NLS), hexadecyltrimethlammonium bromide, or span 80.

### Embodiment 40

The method of any one of the preceding embodiments, wherein said binding buffer is a chaotropic agent comprising an anion selected from the group consisting of thiocyanate, isothiocyanate, perchlorate, acetate, trichloroacetate, trifluoroacetate, chloride, and iodide.

### Embodiment 41

The method of any one of the preceding embodiments, wherein said binding buffer is a chaotropic agent selected from the group consisting of guanidinium hydrochloride (GHCl), guanidinium thiocyanate, guanidinium isothiocyanate (GITC), sodium thiocyanate, sodium iodide, sodium perchlorate, sodium trichloroacetate, sodium trifluroacetate, lithium perchlorate, lithium acetate, magnesium chloride, phenol, 2-propanol, thiourea, and urea.

### Embodiment 42

The method of any one of the preceding embodiments, wherein said binding buffer is a chaotropic agent selected from the group consisting of guanidinium hydrochloride, magnesium chloride, and guanidinium thiocyanate.

### Embodiment 43

The method of any one of the preceding embodiments, wherein the first ATPS composition comprises said polymer at a concentration of 5-80% (w/v), said salt at a concentration of 0.1-80% (w/v), and said surfactant at a concentration of 0-10% (w/v); the volume ratio of the first phase solution to the second phase solution is A:B, and wherein A is 1 and B is 0.9 to 13.

### Embodiment 44

The method of any one of the preceding embodiments, wherein the second ATPS composition comprises said polymer at a concentration of 0.5-30% (w/v), said salt at a concentration of 0.1-10% (w/v), and said surfactant at a concentration of 0-10% (w/v); the volume ratio of the third phase solution to the fourth phase solution is C:D; and wherein C is 1 and D is 1 to 24.

### Embodiment 45

The method of any one of the preceding embodiments, wherein the first ATPS composition comprises 5-80% polymer (w/v) and 0.1-80% salt (w/v); and the second ATPS composition comprises 0.5-30% polymer (w/v) and 5-60% salt (w/v).

### Embodiment 46

The method of any one of the preceding embodiments, wherein the first ATPS composition comprises 5-60% polymer (w/v) and 0.5-50% salt (w/v); and the second ATPS composition comprises 0.5-30% polymer (w/v) and 5-60% salt (w/v).

### Embodiment 47

The method of any one of the preceding embodiments, wherein the first ATPS composition or the second ATPS composition is a polymer-polymer system comprising at least two polymers, and each polymer is dissolved in an aqueous solution at a concentration of 0.2-50% (w/v).

### Embodiment 48

The method of any one of the preceding embodiments, wherein the first ATPS composition or the second ATPS composition is a micellar system comprising one or more surfactants, and each surfactant is dissolved in an aqueous solution at a concentration of 0.1%-90% (w/v).

### Embodiment 49

The method of any one of the preceding embodiments, wherein the first ATPS composition comprises 12-50% polymer (w/v) and 0.1-20% salt (w/v); and the second ATPS composition comprises 0.5-30% polymer (w/v) and 5-60% salt (w/v).

### Embodiment 50

The method of any one of the preceding embodiments, wherein the first ATPS composition further comprises 0.5-2 mM ethylenediaminetetraacetic acid (EDTA), and 0.01-10% surfactant; and the second ATPS composition further comprises 0.5-2 mM EDTA.

### Embodiment 51

The method of any one of the preceding embodiments, wherein the volume ratio between the first phase solution and the second phase solution of the first ATPS composition is A:B. wherein A is 0.1 to 19 and B is 1.

### Embodiment 52

The method of any one of the preceding embodiments, wherein A is 0.9 to 13 and B is 1.

### Embodiment 53

The method of any one of the preceding embodiments, wherein A:B is 13:1, 6:1, or 0.9:1.

### Embodiment 54

The method of any one of the preceding embodiments, wherein the volume ratio between the third phase solution and the fourth phase solution of the second ATPS composition is C:D. wherein C is 1 and D is greater than or equal to 4.

### Embodiment 55

The method of any one of the preceding embodiments, wherein D is 4-100.

### Embodiment 56

The method of any one of the preceding embodiments, wherein D is 24.

### Embodiment 57

The method of any one of the preceding embodiments, wherein A is 5-15; B 1; C is 1; and D is 20-100.

### Embodiment 58

An ATPS composition selected from the group consisting of A1, A2, A3, A4, AA1, AA2, AA3, and AA4.

### Embodiment 59

A method of treating bladder cancer in a patient in need thereof, comprising obtaining a urine sample from the patient, concentrating and purifying at least one target analyte from the urine sample according to the method of any one of the preceding embodiments, analyzing the final solution, and treating the patient with a cancer therapeutic if the target analyte indicates that the patient has bladder cancer or is at risk of developing bladder cancer.

### Embodiment 60

A kit comprising a first ATPS composition selected from the group consisting of A1, A2, A3, and A4; a second ATPS composition selected from the group consisting of AA1, AA2, AA3, and AA4; and a binding buffer selected from the group consisting of B1, B2, and B3.

### Embodiment 61

The kit of any one of the preceding embodiments, further comprising an extraction column.

1. A method for concentrating and purifying one or more target analyte(s) from a bulk fluid sample, comprising the steps of:
(a) dividing the bulk fluid sample containing the target analyte(s) into at least two aliquots of a sample solution;
(b) preparing at least two aliquots of a first aqueous two-phase system (ATPS) composition, wherein the first ATPS composition comprises a polymer, a salt, a surfactant, or any combination thereof dissolved in an aqueous solution to form a first phase solution and a second phase solution;
(c) adding each aliquot of said sample solution prepared from the bulk fluid sample containing the target analyte(s) to each aliquot of the first ATPS composition, such that the target analyte(s) partition to each first phase solution;
(d) further processing each first phase solution to form a final phase solution;
(e) mixing the final phase solution with at least one purifying composition to form a mixed solution;
(f) contacting the mixed solution with a downstream purification system configured to selectively isolate the target analyte(s); and
(g) collecting the target analyte(s) from the downstream purification system, resulting in a final solution containing the concentrated and purified target analyte(s).

2. The method of claim 1, wherein the further processing of step (d) comprises collecting and combining each first phase solution to form the final phase solution.

3. The method of claim 1, wherein the further processing of step (d) comprises the steps of
(i) collecting each first phase solution;
(ii) mixing each first phase solution with a second ATPS composition, wherein the second ATPS composition comprises polymers, salts, surfactants, or combinations thereof dissolved in an aqueous solution to form a third phase solution and a fourth phase solution, such that the target analyte(s) partition to and concentrate in the third phase solution;
(iii) collecting and combining the third phase solutions to form the final phase solution.

4. The method of claim 1, wherein further processing of step (d) comprises the steps of
(i) collecting and combining each first phase solution to form a combined first phase solution;
(ii) mixing each combined first phase solution with a second ATPS composition, wherein the second ATPS composition comprises polymers, salts, surfactants, or combinations thereof dissolved in an aqueous solution to form a third phase solution and a fourth phase solution, such that the target analyte(s) partition to and concentrate in the third phase solution;
(iii) collecting the third phase solution to form the final phase solution.

5. The method of claim 1,
wherein
the purifying composition is a binding buffer comprising at least one chaotropic agent;
the downstream purification system comprises a solid phase medium, wherein the solid phase medium is a solid phase extraction column or a plurality of beads; and
step (f) further comprises the following steps:
(i) contacting a portion of the mixed solution with the solid phase medium such that the target analyte(s) binds to the solid phase medium to form a solid phase extraction complex;
(ii) perturbing the solid phase extraction complex and discarding the flow-through or supernatant; and
(iii) optionally repeating steps (i) and (ii).

6-10. (canceled)

11. The method of claim 1, wherein steps (d), (e), (f), and (g) are the following steps (d1), (e1), (f1), and (g1), respectively:
(d1) collecting the first phase solutions of the at least two aliquots of the first ATPS composition, and mixing the first phase solutions with a second ATPS composition, wherein the second ATPS composition comprises polymers, salts, surfactants, or combinations thereof dissolved in an aqueous solution to form a third phase solution and a fourth phase solution, such that the target analyte(s) partition to and concentrate in the third phase solution;
(e1) collecting the third phase solution and mixing the third phase solution with a binding buffer to form a mixed solution, wherein the binding buffer comprises at least one chaotropic agent;
(f1) loading the mixed solution onto an extraction column configured to selectively extract and purify the target analyte(s); and
(g1) eluting and collecting the target analyte(s) from the extraction column;
wherein the final phase solution of step (d) in claim 1 is the third phase solution of steps (d1) and (e1); the at least one purifying composition of step (e) in claim 1 is the binding buffer of step (e1); and the downstream purification system of steps (f) and (g) in claim 1 is the extraction column of steps (f1) and (g1).

12. The method of claim 1, wherein the bulk fluid sample is selected from the group consisting of blood, plasma, serum, cerebrospinal fluid, urine, saliva, fecal matter, tear, sputum, nasopharyngeal mucus, vaginal discharge and penile discharge.

13. (canceled)

14. The method of claim 1, wherein the bulk fluid sample has a volume of at least 10 mL.

15. (canceled)

16. The method of claim 14, wherein each aliquot of said sample solution has a volume of up to 40 ml.

17-21. (canceled)

22. The method of claim 1, wherein the polymer is dissolved in an aqueous solution at a concentration of 0.2-80% (w/v).

23. The method of claim 1, wherein the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and a copolymer thereof.

24-29. (canceled)

30. The method of claim 1, wherein the salt is dissolved in an aqueous solution at a concentration of 0.1% to 80% (w/v).

31. The method of claim 1, wherein the salt comprises a cation selected from the group consisting of sodium, potassium, calcium, ammonium, lithium, magnesium, aluminium, cesium, barium, straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and an anion selected from the group consisting of phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, hydroxide, hydride, citrate, borate, and tris.

32-36. (canceled)

37. The method of claim 1, wherein the surfactant is dissolved in an aqueous solution at a concentration of 0.05%-10% (w/v).

38. The method of claim 1, wherein the surfactant is selected from the group consisting of anionic surfactant, nonionic surfactant, cationic surfactant, and amphoteric surfactant; and wherein
the anionic surfactant is carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, or sodium N-lauroyl sarcosinate (NLS);
the nonionic surfactant is ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, or polyoxyethylene fatty acid amides;
the cationic surfactant is quaternary ammonium salts, amines with amide linkages, polyoxyethylene alkyl amines, polyoxyethylene alicyclic amines, n,n,n′,n′ tetrakis substituted ethylenediamines, or 2-alkyl 1-hydroxethyl 2-imidazolines; and
the amphoteric surfactant is n-coco 3-aminopropionic acid or sodium salt thereof, n-tallow 3-iminodipropionate or disodium salt thereof, n-carboxymethyl n dimethyl n-9 octadecenyl ammonium hydroxide, or n-cocoamidethyl n hydroxyethylglycine or sodium salt thereof.

39. (canceled)

40. The method of claim 5, wherein said binding buffer is a chaotropic agent comprising an anion selected from the group consisting of thiocyanate, isothiocyanate, perchlorate, acetate, trichloroacetate, trifluoroacetate, chloride, and iodide.

41-50. (canceled)

51. The method of claim 1, wherein the volume ratio between the first phase solution and the second phase solution of the first ATPS composition is A:B, wherein A is 0.1 to 19 and B is 1.

52-53. (canceled)

54. The method of claim 11, wherein the volume ratio between the third phase solution and the fourth phase solution of the second ATPS composition is C:D, wherein C is 1 and D is greater than or equal to 4.

55-58. (canceled)

59. A method of treating bladder cancer in a patient in need thereof, comprising obtaining a urine sample from the patient, concentrating and purifying at least one target analyte from the urine sample according to the method of claim 1, analyzing the final solution, and treating the patient with a cancer therapeutic if the target analyte indicates that the patient has bladder cancer or is at risk of developing bladder cancer.

60. A kit comprising a first ATPS composition selected from the group consisting of A1, A2, A3, and A4; a second ATPS composition selected from the group consisting of AA1, AA2, AA3, and AA4; and a binding buffer selected from the group consisting of B1, B2, and B3.

61. (canceled)
